(19) United States
(12) Patent Application Publication
(10) Pub. No.: US 2024/0189268 A1
(43) Pub. Date: Jun. 13, 2024

(54) SESQUITERPENE DERIVATIVE OR PHARMACEUTICALLY ACCEPTABLE SALT THEREOF AND USE THEREOF

(71) Applicant: MFC CO., LTD., Hwaseong-si, Gyeonggi-do (KR)

(72) Inventors: Hyeon Soo KIM, Seoul (KR); Ji Wook MOON, Seoul (KR); Min Ju KANG, Seoul (KR); Sung Kwan HWANG, Gyeonggi-do (KR); Chang Ha PARK, Gyeonggi-do (KR)

(21) Appl. No.: 18/546,875

(22) PCT Filed: Feb. 17, 2022
(86) PCT No.: PCT/KR2022/002336

§ 371 Date: Nov. 3, 2023

(30) Foreign Application Priority Data
Feb. 18, 2021 (KR) 10-2021-0021526

(51) Int. Cl. A61K 31/216; A23K 20/121; A61K 8/37; A61K 8/49; A61K 31/222; A61K 31/357; A61P 21/00; C07C 63/49; C07C 65/17; C07C 65/26; C07C 69/76; C07C 229/38; C07D 317/38

(52) U.S. Cl. CPC A61K 31/216; A23K 20/121; A61K 8/37; A61K 8/49; A61K 31/222; A61K 31/357; A61P 21/00; C07C 63/49; C07C 65/17; C07C 65/26; C07C 69/76; C07C 229/38; C07D 317/38; C07C 2603/86

(57) ABSTRACT

Described herein is a sesquiterpene derivative or a pharmaceutically acceptable salt thereof, a composition for preventing, ameliorating or treating sarcopenia, including the derivative or salt thereof as an active ingredient, and the like. The sesquiterpene derivative or pharmaceutically acceptable salt thereof inhibits increases in the production and mRNA expression of a myostatin protein, which directly affects muscle loss and reduced muscle strength, and thus may exhibit a more fundamental effect of preventing or treating sarcopenia.

### TECHNICAL FIELD

The present disclosure relates to a sesquiterpene derivative or a pharmaceutically acceptable salt, uses thereof, and the like.

### BACKGROUND ART

The concept of sarcopenia began in 1989 when Irwin Rosenberg introduced the term “sarcopenia”, and is a combination of the words “sarco” meaning muscle and “penia” meaning decreased, in its origins in Greek. The sarcopenia is associated with aging and refers to reduced muscle strength due to a decrease in muscle mass. Here, “muscle” refers to skeletal muscle and has nothing to do with smooth muscle. In other words, the sarcopenia refers to a loss of skeletal muscle mass mainly distributed in the limbs, and is distinguished from cachexia, a state of significant muscle loss that occurs in the late stage of malignant tumors, muscle wasting due to acute diseases such as the flu, or primary muscle disease.

Recently, as the age group of 65 years or older has rapidly increased, the prevalence of osteoporosis and sarcopenia is also rapidly increasing. It is estimated that a gradual decrease in muscle mass occurs after the age of 40, and a decrease of 8% in the muscle mass occurs every 10 years until the age of 70, and thereafter, it is known that a more rapid decrease in the muscle mass occurs, and the decrease in the muscle mass may occur up to 15% every 10 years. Through many follow-up studies, it has been found that the physiological changes that occur in the elderly are diverse, and in general, the muscle mass and the bone density decrease simultaneously with increasing age.

Meanwhile, there may be three major treatment methods for sarcopenia. The first is exercise. It has been reported that the exercise increases the protein synthesis ability of skeletal muscle in the short term and increases muscle strength or mobility in the elderly. However, the exercise is not suitable for long-term treatment. Second, testosterone or anabolic steroids may be used as drug treatment but induces masculinization in women and causes side effects such as prostate symptoms in men. Other approved prescriptions include dehydroepiandrosterone (DHEA) and growth hormone, which have been reported in studies that may be used as treatment methods at sites containing selective androgen receptor modulators (SARMs). In addition, although diet therapy is known as a treatment method, according to nutritional assessments, malnutrition, and modern eating habits are inadequate to maintain adequate total body mass.

Myostatin is a polypeptide growth factor belonging to a TGF-β superfamily. TGF-β has a large number of isoforms, which are known to be involved in cell proliferation, apoptosis, differentiation, and bone formation and maintenance (Massague & Chen, 2000). The myostatin belongs to growth differentiation factor (GDF) No. 8, is involved in tissue growth and development, and acts by activating a Smad signaling system. In addition, it has been reported to affect bone formation and regeneration by inhibiting the cell cycle and proliferation of progenitor cells by a p21 gene. The myostatin is mainly produced in skeletal muscle cells and causes muscle loss and reduced muscle strength in an autocrine manner, and is known to inhibit the expression of IGF-1 or Follistatin, which is involved in muscle hypertrophy, thereby inhibiting protein synthesis and cell proliferation in myoblasts.

Under the background, the present inventors have made many efforts to discover a substance capable of treating sarcopenia by inhibiting the expression of myostatin causing muscle loss and reduced muscle strength and promoter activity, and as a result, found that a sesquiterpene derivative ingredient is able to be used to prevent or treat sarcopenia by inhibiting the expression of myostatin, and then completed the present disclosure.

### DISCLOSURE OF THE INVENTION

### Technical Goals

An aspect of the present disclosure is to provide a sesquiterpene derivative or a pharmaceutically acceptable salt thereof.

Another aspect of the present disclosure is to provide a pharmaceutical composition for preventing or treating sarcopenia including the derivative or a pharmaceutically acceptable salt thereof as an active ingredient.

Yet another aspect of the present disclosure is to provide a cosmetic composition for preventing or ameliorating sarcopenia including the derivative or a cosmetically acceptable salt thereof as an active ingredient.

Yet another aspect of the present disclosure is to provide a food composition for preventing or ameliorating sarcopenia including the derivative or a food acceptable salt thereof as an active ingredient.

Yet another aspect of the present disclosure is to provide a feed composition for preventing or ameliorating sarcopenia including the derivative or a feed acceptable salt thereof as an active ingredient.

However, technical goals to be achieved are not limited to those described above. and other goals not mentioned above are clearly understood by one of ordinary skill in the art from the following description.

### Technical Solutions

According to an embodiment of the present disclosure, there is provided a sesquiterpene derivative represented by Chemical Formula 1 below or a pharmaceutically acceptable salt thereof.

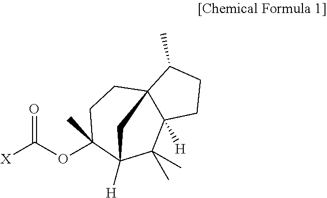

As an embodiment of the present disclosure, in Chemical Formula 1, X is

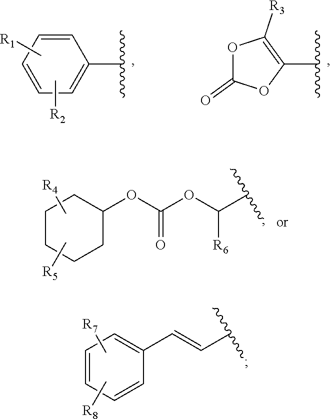

R₁ to R₈ are each independently hydrogen, halogen, straight or branched C1-C5 alkyl, straight or branched C2-C5 alkenyl, C2-C5 alkynyl, C3-C7 cycloalkyl, straight or branched C1-C5 alkoxy, —C(═O)Y₁, —OC(═O)Y₂, cyano, or hydroxyl (wherein one or more hydrogens of the C1-C5 alkyl, C3-C7 cycloalkyl, or C1-C5 alkoxy are each independently unsubstituted or substituted with one or more substituents selected from the group consisting of halogen, hydroxy, cyano, nitro, NY₃Y₄, and

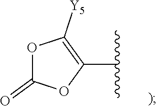

Y₁ to Y₅ are each independently hydrogen, straight or branched C1-C5 alkyl, or C3-C7 cycloalkyl (wherein one or more hydrogens of the C1-C5 alkyl or C3-C7 cycloalkyl are independently unsubstituted or substituted with one or more substituents selected from the group consisting of halogen, hydroxy, cyano, nitro, NZ₁Z₂, and

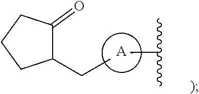

Z₁ and Z₂ are each independently hydrogen or straight or branched C1-C5 alkyl; and A is a C3-C7 aryl group or a C3-C7 heteroaryl group.

However, there are excluded 1) (3R,3aS,6R,7R,8aS)-3,6,8,8-tetramethyloctahydro-1H-3a,7-metanoazulen-6-yl-4-hydroxy-3- methoxybenzoate;
2) (3R,3aS,6R,7R,8aS)-3,6,8,8-tetramethyloctahydro-1H-3a,7-metanoazulen-6-yl-4-acetoxy-3- methoxybenzoate;
3) (3R,3aS,6R,7R,8aS)-3,6,8,8-tetramethyloctahydro-1H-3a,7-methanoazulen-6-yl-3-methoxy-4-pivaloyloxybenzoate;
4) (3R,3aS,6R,7R,8aS)-3,6,8,8-tetramethyloctahydro-1H-3a,7-methanoazulen-6-yl-5-methyl-2-oxo-1,3-dioxole-4-carboxylate;
5) (3R,3aS,6R,7R,8aS)-3,6,8,8-tetramethyloctahydro-1H-3a,7-methanoazulen-6-yl-2-(((cyclohexyloxy)carbonyl)oxy)propanate;
6) (3R,3aS,6R,7R,8aS)-3,6,8,8-tetramethyloctahydro-1H-3a,7-metanoazulen-6-yl-4-(pivaloyloxy)benzoate;
7) (E)-(3R,3aS,6R,7R,8aS)-3,6,8,8-tetramethyloctahydro-1H-3a,7-methanoazulen-6-yl-3-(4-hydroxy phenyl)acrylate;
8) (E)-(3R,3aS,6R,7R,8aS)-3,6,8,8-tetramethyloctahydro-1H-3a,7-methanoazulen-6-yl-3-(4-hydroxy-3-methoxyphenyl)acrylate;
9) (E)-(3R,3aS,6R,7R,8aS)-3,6,8,8-tetramethyloctahydro-1H-3a,7-methanoazulen -6-yl-3-(3,4-dihydroxy phenyl)acrylate;
10) (E)-(3R,3aS,6R,7R,8aS)-3,6,8,8-tetramethyloctahydro-1H-3a,7-methanoazulen-6-yl cinnamate;
11) (E)-(3R,3aS,6R,7R,8aS)-3,6,8,8-tetramethyloctahydro-1H-3a,7-methanoazulen-6-yl-3-(4-(2-hydroxyethoxy)phenypacrylate;
12) (E)-(3R,3aS,6R,7R,8aS)-3,6,8,8-tetramethyloctahydro-1H-3a,7-methanoazulen-6-yl-3-(4-(2-(dimethylamino)ethoxy)phenyl)acrylate.

As another embodiment of the present disclosure, the sesquiterpene derivative represented by Chemical Formula 1 above may be any one or more selected from the group consisting of the following compounds 13) to 29).
13) (3R,3aS,6R,7R,8aS)-3,6,8,8-tetramethyloctahydro-1H-3a,7-methanoazulen-6-yl benzoate;
14) (3R,3aS,6R,7R,8aS)-3,6,8,8-tetramethyloctahydro-1H-3a,7-methanoazulen-6-yl-4-hydroxybenzoate;
15) (3R,3aS,6R,7R,8aS)-3,6,8,8-tetramethyloctahydro-1H-3a,7-methanoazulen-6-yl 4-acetoxybenzoate;
16) (3R,3aS,6R,7R,8aS)-3,6,8,8-tetramethyloctahydro-1H-3a,7-methanoazulen-6-yl 4-(2-hydroxyethoxy)benzoate;
17) (3R,3aS,6R,7R,8aS)-3,6,8,8-tetramethyloctahydro-1H-3a,7-methanoazulen-6-yl 4-(2-dimethylamino)ethoxy)benzoate;
18) (3R,3aS,6R,7R,8aS)-3,6,8,8-tetramethyloctahydro-1H-3a,7-methanoazulen-6-yl 4-((cyclopentanecarbonyl)oxy)benzoate;
19) (3R,3aS,6R,7R,8aS)-3,6,8,8-tetramethyloctahydro-1H-3a,7-methanoazulen-6-yl 4-((cyclohexanecarbonyl)oxy)benzoate;
20) (3R,3aS,6R,7R,8aS)-3,6,8,8-tetramethyloctahydro-1H-3a,7-methanoazulen-6-yl 4-(((S)-2-amino-4-methylpentanoyl)oxy)benzoate;
21) (E)-(3R,3aS,6R,7R,8aS)-3,6,8,8-tetramethyloctahydro-1H-3a,7-methanoazulen-6-yl-3-(4-methoxy) cinnamate;
22) (E)-(3R,3aS,6R,7R,8aS)-3,6,8,8-tetramethyloctahydro-1H-3a,7-methanoazulen-6-yl-3-(4-(pivaloyloxy)phenyl)acrylate;
23) (E)-(3R,3aS,6R,7R,8aS)-3,6,8,8-tetramethyloctahydro-1H-3a,7-methanoazulen-6-yl-3-(4-medoxomiloxy)phenyl)acrylate;
24) (E)-(3R,3aS,6R,7R,8aS)-3,6,8,8-tetramethyloctahydro-1H-3a,7-methanoazulen-6-yl-3-(3-methoxy-4-pivaloyloxy)phenypacrylate;
25) (E)-(3R,3aS,6R,7R,8aS)-3,6,8,8-tetramethyloctahydro-1H-3a,7-methanoazulen-6-yl-3-(3-hydroxy-4-pivaloyloxy)phenyl)acrylate;
26) 2-methoxy-5-((E)-3-oxo-3-(((3R,3aS,6R,7R,8aS)-3,6,8,8-tetramethyloctahydro-1H-3a,7-methanoazulen-6-yl)oxy)propen-1-yl)phenyl L-leucinate;
27) (3R,3aS,6R,7R,8aS)-3,6,8,8-tetramethyloctahydro-1H-3a,7-methanoazulen-6-yl (E)-3-(4-acetoxy-3-methoxyphenyl)acrylate;
28) (3R,3aS,6R,7R,8aS)-3,6,8,8-tetramethyloctahydro-1H-3a,7-methanoazulen-6-yl (E)-3-(4-isopropoxy-3-methoxyphenyl)acrylate; and
29) (3R,3aS,6R,7R,8aS)-3,6,8,8-tetramethyloctahydro-1H-3a,7-methanoazulen-6-yl (E)-3-(3-methoxy-4-((2-(4-((2-oxocyclopentyl)phenyl)propanoyl)oxy)phenyl)acrylate.

According to another embodiment of the present disclosure, there is provided a pharmaceutical composition for preventing or treating sarcopenia including a sesquiterpene derivative represented by Chemical Formula 1 below or a pharmaceutically acceptable salt thereof as an active ingredient.

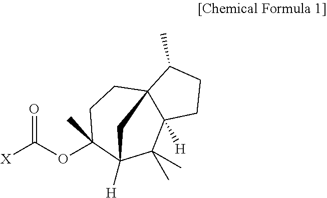

As an embodiment of the present disclosure, in Chemical Formula 1, X is

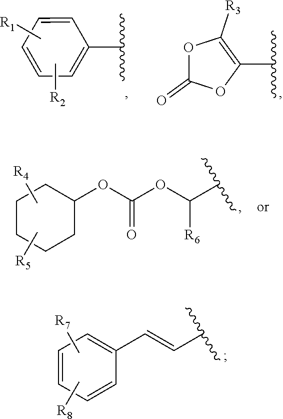

R₁ to R₈ are each independently hydrogen, halogen, straight or branched C1-C5 alkyl, straight or branched C2-C5 alkenyl, C2-C5 alkynyl, C3-C7 cycloalkyl, straight or branched C1-C5 alkoxy, —C(═O)Y₁, —OC(═O)Y₂, cyano, or hydroxyl (wherein one or more hydrogens of the C1-C5 alkyl, C3-C7 cycloalkyl, or C1-C5 alkoxy are each independently unsubstituted or substituted with one or more substituents selected from the group consisting of halogen, hydroxy, cyano, nitro, NY₃Y₄, and

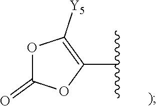

Y₁ to Y₅ are each independently hydrogen, straight or branched C1-C5 alkyl, or C3-C7 cycloalkyl (wherein one or more hydrogens of the C1-C5 alkyl or C3-C7 cycloalkyl are independently unsubstituted or substituted with one or more substituents selected from the group consisting of halogen, hydroxy, cyano, nitro, NZ₁Z₂, and

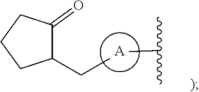

Z₁ and Z₂ are each independently hydrogen or straight or branched C1-C5 alkyl; and A is a C3-C7 aryl group or a C3-C7 heteroaryl group.

According to yet another embodiment of the present disclosure, there is provided a method for preventing or treating sarcopenia, including administering the sesquiterpene derivative or a pharmaceutically acceptable salt thereof to a subject.

According to yet another embodiment of the present disclosure, there is provided a use of the sesquiterpene derivative or a pharmaceutically acceptable salt thereof for preparation of a drug for preventing or treating sarcopenia.

As an embodiment of the present disclosure, the sesquiterpene derivative or the pharmaceutically acceptable salt thereof may inhibit the expression of myostatin mRNA or protein.

As another embodiment of the present disclosure, the sesquiterpene derivative or the pharmaceutically acceptable salt thereof may inhibit the expression of Muscle RING-finger protein-1 (MURF1) mRNA or protein or inhibit the expression of forkhead box O3 (Foxo3) mRNA or protein.

As another embodiment of the present disclosure, the sesquiterpene derivative represented by Chemical Formula 1 above may be any one or more selected from the group consisting of the following compounds 1) to 29).
1) (3R,3aS,6R,7R,8aS)-3,6,8,8-tetramethyloctahydro-1H-3a,7-metanoazulen-6-yl-4-hydroxy-3-methoxybenzoate;
2) (3R,3aS,6R,7R,8aS)-3,6,8,8-tetramethyloctahydro-1H-3a,7-metanoazulen-6-yl-4-acetoxy-3- methoxybenzoate;
3) (3R,3aS,6R,7R,8aS)-3,6,8,8-tetramethyloctahydro-1H-3a,7-methanoazulen-6-yl-3-methoxy-4-pivaloyloxybenzoate;
4) (3R,3aS,6R,7R,8aS)-3,6,8,8-tetramethyloctahydro-1H-3a,7-methanoazulen-6-yl-5-methyl-2-oxo-1,3-dioxole-4-carboxylate;
5) (3R,3aS,6R,7R,8aS)-3,6,8,8-tetramethyloctahydro-1H-3a,7-methanoazulen-6-yl-2-(((cyclohexyloxy)carbonyl)oxy)propanate;
6) (3R,3aS,6R,7R,8aS)-3,6,8,8-tetramethyloctahydro-1H-3a,7-metanoazulen-6-yl-4-(pivaloyloxy)benzoate;
7) (E)-(3R,3aS,6R,7R,8aS)-3,6,8,8-tetramethyloctahydro-1H-3a,7-methanoazulen-6-yl-3-(4-hydroxy phenyl)acrylate;
8) (E)-(3R,3aS,6R,7R,8aS)-3,6,8,8-tetramethyloctahydro-1H-3a,7-methanoazulen-6-yl-3-(4-hydroxy-3-methoxyphenyl)acrylate;
9) (E)-(3R,3aS,6R,7R,8aS)-3,6,8,8-tetramethyloctahydro-1H-3a,7-methanoazulen-6-yl-3-(3,4-dihydroxy phenyl)acrylate;
10) (E)-(3R,3aS,6R,7R,8aS)-3,6,8,8-tetramethyloctahydro-1H-3a,7-methanoazulen-6-yl cinnamate;
11) (E)-(3R,3aS,6R,7R,8aS)-3,6,8,8-tetramethyloctahydro-1H-3a,7-methanoazulen-6-yl-3-(4-(2-hydroxyethoxy)phenyl)acrylate;
12) (E)-(3R,3aS,6R,7R,8aS)-3,6,8,8-tetramethyloctahydro-1H-3a,7-methanoazulen-6-yl-3-(4-(2-(dimethylamino)ethoxy)phenypacrylate;
13) (3R,3aS,6R,7R,8aS)-3,6,8,8-tetramethyloctahydro-1H-3a,7-methanoazulen-6-yl benzoate;
14) (3R,3aS,6R,7R,8aS)-3,6,8,8-tetramethyloctahydro-1H-3a,7-methanoazulen-6-yl-4-hydroxybenzoate;
15) (3R,3aS,6R,7R,8aS)-3,6,8,8-tetramethyloctahydro-1H-3a,7-methanoazulen-6-yl 4-acetoxybenzoate;
16) (3R,3aS,6R,7R,8aS)-3,6,8,8-tetramethyloctahydro-1H-3a,7-methanoazulen-6-yl 4-(2-hydroxyethoxy)benzoate;
17) (3R,3aS,6R,7R,8aS)-3,6,8,8-tetramethyloctahydro-1H-3a,7-methanoazulen-6-yl 4-(2-dimethylamino)ethoxy)benzoate;
18) (3R,3aS,6R,7R,8aS)-3,6,8,8-tetramethyloctahydro-1H-3a,7-methanoazulen-6-yl 4-((cyclopentanecarbonyl)oxy)benzoate;
19) (3R,3aS,6R,7R,8aS)-3,6,8,8-tetramethyloctahydro-1H-3a,7-methanoazulen-6-yl 4-((cyclohexanecarbonyl)oxy)benzoate;
20) (3R,3aS,6R,7R,8aS)-3,6,8,8-tetramethyloctahydro-1H-3a,7-methanoazulen-6-yl 4-(((S)-2-amino-4-methylpentanoyl)oxy)benzoate;
21) (E)-(3R,3aS,6R,7R,8aS)-3,6,8,8-tetramethyloctahydro-1H-3a,7-methanoazulen-6-yl-3-(4-methoxy) cinnamate;
22) (E)-(3R,3aS,6R,7R,8aS)-3,6,8,8-tetramethyloctahydro-1H-3a,7-methanoazulen-6-yl-3-(4-(pivaloyloxy)phenyl)acrylate;
23) (E)-(3R,3aS,6R,7R,8aS)-3,6,8,8-tetramethyloctahydro-1H-3a,7-methanoazulen-6-yl-3-(4-medoxomiloxy)phenyl)acrylate;
24) (E)-(3R,3aS,6R,7R,8aS)-3,6,8,8-tetramethyloctahydro-1H-3a,7-methanoazulen-6-yl-3-(3-methoxy-4-pivaloyloxy)phenyl)acrylate;
25) (E)-(3R,3aS,6R,7R,8aS)-3,6,8,8-tetramethyloctahydro-1H-3a,7-methanoazulen-6-yl-3-(3-hydroxy-4-pivaloyloxy)phenyl)acrylate;
26) 2-methoxy-5-((E)-3-oxo-3-(((3R,3aS ,6R,7R,8aS)-3,6,8,8-tetramethyloctahydro-1H-3a,7-methanoazulen-6-yl)oxy)propen-1-yl)phenyl L-leucinate;
27) (3R,3aS,6R,7R,8aS)-3,6,8,8-tetramethyloctahydro-1H-3a,7-methanoazulen-6-yl (E)-3-(4-acetoxy-3-methoxyphenyl)acrylate;
28) (3R,3aS,6R,7R,8aS)-3,6,8,8-tetramethyloctahydro-1H-3a,7-methanoazulen-6-yl (E)-3-(4-isopropoxy-3-methoxyphenyl)acrylate; and
29) (3R,3aS,6R,7R,8aS)-3,6,8,8-tetramethyloctahydro-1H-3a,7-methanoazulen-6-yl (E)-3-(3-methoxy-4-((2-(4-((2-oxocyclopentyl)phenyl)propanoyl)oxy)phenyl)acrylate.

As another embodiment of the present disclosure, the composition for preventing or treating the sarcopenia may include one or more additional ingredients selected from the group consisting of pharmaceutically acceptable carriers, excipients, diluents, stabilizers and preservatives, and the sesquiterpene derivative or a pharmaceutically acceptable salt thereof.

As yet another embodiment of the present disclosure, the pharmaceutical composition may have formulations of powders, granules, tablets, capsules, or injections.

According to yet another embodiment of the present disclosure, there is provided a cosmetic composition for preventing or ameliorating sarcopenia including the sesquiterpene derivative or a cosmetically acceptable salt thereof as an active ingredient.

According to yet another embodiment of the present disclosure, there is provided a food composition for preventing or ameliorating sarcopenia including the sesquiterpene derivative or a food acceptable salt thereof as an active ingredient.

According to yet another embodiment of the present disclosure, there is provided a feed composition for preventing or ameliorating sarcopenia including the sesquiterpene derivative or a feed acceptable salt thereof as an active ingredient.

### Effects

The present disclosure relates to a sesquiterpene derivative or a pharmaceutically acceptable salt thereof, a composition for preventing, ameliorating or treating sarcopenia, including the derivative or salt thereof as an active ingredient. The sesquiterpene derivative or pharmaceutically acceptable salt thereof of the present disclosure inhibits increases in the production and mRNA expression of a myostatin protein which directly affects muscle loss and reduced muscle strength, and thus may exhibit a more fundamental effect of preventing or treating sarcopenia.

The effects of the present disclosure are not limited to the aforementioned effects, and other objects, which are not mentioned above, will be clearly appreciated by a person having ordinary skill in the art from the following description.

### BRIEF DESCRIPTION OF DRAWINGS

FIG. 1 is a diagram illustrating results of an experiment on an effect of sesquiterpene derivatives on muscle loss inhibition according to compound type and content. FIG. 1A illustrates results of cell viability by concentration of Cedrol (MFC Reference), Compound 1-1 (MFC-1), and Compound 1-2 (MFC-2). FIGS. 1B and 1C illustrate results of luciferase activity of Compound 1-1 (MFC-1) and Compound 1-2 (MFC-2), respectively.

FIG. 2 is a diagram illustrating results of an experiment on an effect of sesquiterpene derivatives on muscle loss inhibition according to compound type and content. FIGS. 2D and 2E confirm the expression of myostatin and MURF-1, respectively, upon UV irradiation after pretreatment with Cedrol (MFC Reference), Compound 1-1 (MFC-1), and Compound 1-2 (MFC-2). FIG. 2F confirms the expression of myostatin upon pretreatment with Cedrol (MFC-0), Compound 1-1 (MFC-1), and Compound 1-2 (MFC-2). FIG. 2G confirms the expression of myostatin, upon treatment of doxorubicin after pretreatment with Cedrol (MFC-0), Compound 1-1 (MFC-1), and Compound 1-2 (MFC-2).

FIG. 3 is a diagram illustrating changes in Luciferase activity according to Compound 1-2 (MFC-2). FIG. 3H confirms promoter activity of pNF-κB after pretreatment with Compound 1-2 (MFC-2). FIG. 3I confirms promoter activity of NF-κB upon treatment of doxorubicin after pretreatment with Compound 1-2 (MFC-2). FIG. 3J illustrates pMSTN-luciferase activity results of Cedrol (MFC Reference) and Compound 1-2 (MFC-2).

FIG. 4 is a diagram illustrating results of muscle analysis of aging mice treated with regular feed (Control), Cedrol (Reference), and Compound 1-2 (MFC-2). FIG. 4A illustrates Gastrocnemius muscle (GC), Tibialis anterior (TA), and Extensor digitorum longus (EDL) of aging mice. FIG. 4B illustrates results of measuring grip strength. FIG. 4C illustrates the expression of EDL myostatin and EDL MuRF1.

FIG. 5 is a diagram illustrating results of blood analysis of aging mice treated with regular feed (Control), Cedrol (Reference), and Compound 1-2 (MFC-2). FIGS. 5A to 5D illustrate results of creatine kinase (CK), lactate dehydrogenase (LDH), aspartate transaminase (AST), and triglyceride (TG), respectively.

### BEST MODE FOR CARRYING OUT THE INVENTION

Hereinafter, embodiments will be described in detail with reference to the accompanying drawings. However, since various modifications may be made to embodiments, the scope of the present disclosure is not limited or restricted by these embodiments. It should be understood that all modifications, equivalents and substitutes for embodiments are included in the scope of the present disclosure.

The terms used in the embodiments are used for the purpose of description only, and should not be construed to be limited. The singular expression includes the plural expression unless the context clearly dictates otherwise. In the present disclosure, it should be understood that term “including” or “having” indicates that a feature, a number, a step, an operation, a component, a part or the combination thereof described in the specification is present, but does not exclude a possibility of presence or addition of one or more other features, numbers, steps, operations, components, parts or combinations thereof, in advance.

Unless otherwise contrarily defined, all terms used herein including technological or scientific terms have the same meanings as those generally understood by a person with ordinary skill in the art to which embodiments pertain. Terms which are defined in a generally used dictionary should be interpreted to have the same meaning as the meaning in the context of the related art, and are not interpreted as ideal or excessively formal meanings unless otherwise defined in the present disclosure.

In addition, in the description with reference to the accompanying drawings, like components designate like reference numerals regardless of reference numerals and a duplicated description thereof will be omitted. In describing the embodiments, a detailed description of related known technologies will be omitted if it is determined that they unnecessarily make the gist of the embodiments unclear.

According to an embodiment of the present disclosure, there is provided a sesquiterpene derivative or a pharmaceutically acceptable salt thereof.

Specifically, the sesquiterpene derivative may be a compound represented by Chemical Formula 1 below, or a racemate, isomer, or solvate thereof.

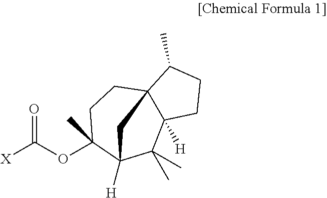

In Chemical Formula 1, X is

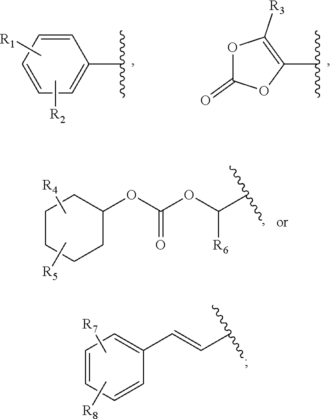

R₁ to R₈ are each independently hydrogen, halogen, straight or branched C1-C5 alkyl, straight or branched C2-C5 alkenyl, C2-C5 alkynyl, C3-C7 cycloalkyl, straight or branched C1-C5 alkoxy, —C(═O)Y₁, —OC(═O)Y₂, cyano, or hydroxyl (wherein one or more hydrogens of the C1-C5 alkyl, C3-C7 cycloalkyl, or C1-C5 alkoxy are each independently unsubstituted or substituted with one or more substituents selected from the group consisting of halogen, hydroxy, cyano, nitro, NY₃Y₄, and

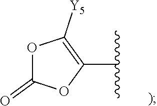

Y₁ to Y₅ are each independently hydrogen, straight or branched C1-C5 alkyl, or C3-C7 cycloalkyl (wherein one or more hydrogens of the C1-C5 alkyl or C3-C7 cycloalkyl are independently unsubstituted or substituted with one or more substituents selected from the group consisting of halogen, hydroxy, cyano, nitro, NZ₁Z₂, and

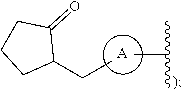

Z₁ and Z₂ are each independently hydrogen or straight or branched C1-C5 alkyl; and A is a C3-C7 aryl group or a C3-C7 heteroaryl group.

More specifically, the sesquiterpene derivative used as the pharmaceutical composition for preventing or treating sarcopenia may be one or more of the following compounds 1) to 29):
1) (3R,3aS,6R,7R,8aS)-3,6,8,8-tetramethyloctahydro-1H-3a,7-metanoazulen-6-yl-4-hydroxy-3-methoxybenzoate;
2) (3R,3aS,6R,7R,8aS)-3,6,8,8-tetramethyloctahydro-1H-3a,7-metanoazulen-6-yl-4-acetoxy-3-methoxybenzoate;
3) (3R,3aS,6R,7R,8aS)-3,6,8,8-tetramethyloctahydro-1H-3a,7-methanoazulen-6-yl-3-methoxy-4-pivaloyloxybenzoate;
4) (3R,3aS,6R,7R,8aS)-3,6,8,8-tetramethyloctahydro-1H-3a,7-methanoazulen-6-yl-5-methyl-2-oxo-1,3-dioxole-4-carboxylate;
5) (3R,3aS,6R,7R,8aS)-3,6,8,8-tetramethyloctahydro-1H-3a,7-methanoazulen-6-yl-2-(((cyclohexyloxy)carbonyl)oxy)propanate;
6) (3R,3aS,6R,7R,8aS)-3,6,8,8-tetramethyloctahydro-1H-3a,7-metanoazulen-6-yl-4-(pivaloyloxy)benzoate;
7) (E)-(3R,3aS,6R,7R,8aS)-3,6,8,8-tetramethyloctahydro-1H-3a,7-methanoazulen-6-yl-3-(4-hydroxy phenyl)acrylate;
8) (E)-(3R,3aS,6R,7R,8aS)-3,6,8,8-tetramethyloctahydro-1H-3a,7-methanoazulen-6-yl-3-(4-hydroxy-3-methoxyphenyl)acrylate;
9) (E)-(3R,3aS,6R,7R,8aS)-3,6,8,8-tetramethyloctahydro-1H-3a,7-methanoazulen-6-yl-3-(3,4-dihydroxyphenyl)acrylate;
10) (E)-(3R,3aS,6R,7R,8aS)-3,6,8,8-tetramethyloctahydro-1H-3a,7-methanoazulen-6-yl cinnamate;
11) (E)-(3R,3aS,6R,7R,8aS)-3,6,8,8-tetramethyloctahydro-1H-3a,7-methanoazulen-6-yl-3-(4-(2-hydroxyethoxy)phenypacrylate;
12) (E)-(3R,3aS,6R,7R,8aS)-3,6,8,8-tetramethyloctahydro-1H-3a,7-methanoazulen-6-yl-3-(4-(2-(dimethylamino)ethoxy)phenyl)acrylate;
13) (3R,3aS,6R,7R,8aS)-3,6,8,8-tetramethyloctahydro-1H-3a,7-methanoazulen-6-yl benzoate;
14) (3R,3aS,6R,7R,8aS)-3,6,8,8-tetramethyloctahydro-1H-3a,7-methanoazulen-6-yl-4-hydroxybenzoate;
15) (3R,3aS,6R,7R,8aS)-3,6,8,8-tetramethyloctahydro-1H-3a,7-methanoazulen-6-yl 4-acetoxybenzoate;
16) (3R,3aS,6R,7R,8aS)-3,6,8,8-tetramethyloctahydro-1H-3a,7-methanoazulen-6-yl 4-(2-hydroxyethoxy)benzoate;
17) (3R,3aS,6R,7R,8aS)-3,6,8,8-tetramethyloctahydro-1H-3a,7-methanoazulen-6-yl 4-(2-dimethylamino)ethoxy)benzoate;
18) (3R,3aS,6R,7R,8aS)-3,6,8,8-tetramethyloctahydro-1H-3a,7-methanoazulen-6-yl 4-((cyclopentanecarbonyl)oxy)benzoate;
19) (3R,3aS,6R,7R,8aS)-3,6,8,8-tetramethyloctahydro-1H-3a,7-methanoazulen-6-yl 4-((cyclohexanecarbonyl)oxy)benzoate;
20) (3R,3aS,6R,7R,8aS)-3,6,8,8-tetramethyloctahydro-1H-3a,7-methanoazulen-6-yl 4-(((S)-2-amino-4-methylpentanoyl)oxy)benzoate;
21) (E)-(3R,3aS,6R,7R,8aS)-3,6,8,8-tetramethyloctahydro-1H-3a,7-methanoazulen-6-yl-3-(4-methoxy) cinnamate;
22) (E)-(3R,3aS,6R,7R,8aS)-3,6,8,8-tetramethyloctahydro-1H-3a,7-methanoazulen-6-yl-3-(4-(pivaloyloxy)phenyl)acrylate;
23) (E)-(3R,3aS,6R,7R,8aS)-3,6,8,8-tetramethyloctahydro-1H-3a,7-methanoazulen-6-yl-3-(4-medoxomiloxy)phenyl)acrylate;
24) (E)-(3R,3aS,6R,7R,8aS)-3,6,8,8-tetramethyloctahydro-1H-3a,7-methanoazulen-6-yl-3-(3 -methoxy-4-pivaloyloxy)phenyl)acrylate;
25) (E)-(3R,3aS,6R,7R,8aS)-3,6,8,8-tetramethyloctahydro-1H-3a,7-methanoazulen-6-yl-3-(3-hydroxy-4-pivaloyloxy)phenyl)acrylate;
26) 2-methoxy-5-((E)-3-oxo-3-(((3R,3aS,6R,7R,8aS)-3,6,8,8-tetramethyloctahydro-1H-3a,7-methanoazulen-6-yl)oxy)propen-1-yl)phenyl L-leucinate;
27) (3R,3aS,6R,7R,8aS)-3,6,8,8-tetramethyloctahydro-1H-3a,7-methanoazulen-6-yl (E)-3-(4-acetoxy-3-methoxyphenyl)acrylate;
28) (3R,3aS,6R,7R,8aS)-3,6,8,8-tetramethyloctahydro-1H-3a,7-methanoazulen-6-yl (E)-3-(4-isopropoxy-3-methoxyphenyl)acrylate; and
29) (3R,3aS,6R,7R,8aS)-3,6,8,8-tetramethyloctahydro-1H-3a,7-methanoazulen-6-yl (E)-3-(3-methoxy-4-((2-(4-((2-oxocyclopentyl)phenyl)propanoyl)oxy)phenyl)acrylate.

As used herein, the terms used to define substituents of the compound represented by Chemical Formula 1 or the pharmaceutically acceptable salt thereof are as follows.

As used herein, the term “substitution” means that a hydrogen atom bound to a carbon atom of a compound is replaced with another substituent, wherein a position to be substituted is not limited as long as the position is a position where a hydrogen atom is substituted, that is, a position where a substituent may be substituted, and when two or more substituents are substituted, the two or more substituents may be the same as or different from each other.

As used herein, the term “halogen” refers to a halogen group element and includes, for example, fluoro (F), chloro (CO, bromo (Br), or iodine (I).

As used herein, the term “alkyl” refers to a saturated hydrocarbon having carbon atoms. Examples of alkyl include, without limitation, methyl, ethyl, propyl, and butyl.

As used herein, the term “alkenyl” refers to an unsaturated hydrocarbon having a carbon atoms containing at least one double bond. Examples of alkenyl include, without limitation, ethenyl, propenyl, and butenyl.

As used herein, the term “alkynyl” refers to an unsaturated hydrocarbon having carbon atoms containing at least one triple bond. Examples of alkynyl include, without limitation, ethynyl, propynyl, and butynyl.

As used herein, the term “cycloalkyl” refers to a cyclic saturated hydrocarbon having carbon atoms. Examples of cycloalkyl include, without limitation, cyclopropyl, cyclobutyl, cyclopentyl, cyclohexyl, and cycloheptyl.

As used herein, the term “alkoxy” refers to a monovalent atomic group in which alkyl is bonded to oxygens. Examples of alkoxy include, without limitation, methoxy, ethoxy, propoxy, and butoxy.

As used herein, the term “pharmaceutically acceptable” may mean that the composition is physiologically acceptable and does not generally cause an allergic reaction, such as gastrointestinal disorder, and dizziness, or similar reactions thereto when administered to humans.

In addition, the pharmaceutically acceptable salt may be prepared by conventional methods known to those skilled in the art, and may correspond to forms of acid addition salts or base addition salts suitable or compatible for the treatment of patients herein. Examples of inorganic acids which form suitable salts may include not only hydrochloric, hydrobromic, sulfuric and phosphoric acids, but also metal salts, such as sodium monohydrogen orthophosphate and potassium hydrogen sulfate. Examples of organic acids which form suitable salts may include not only mono-, di- and tricarboxylic acids, such as glycolic acid, lactic acid, pyruvic acid, malonic acid, succinic acid, glutaric acid, fumaric acid, malic acid, tartaric acid, citric acid, ascorbic acid, maleic acid, benzoic acid, phenylacetic acid, cinnamic acid and salicylic acid, but also sulfonic acids, such as p-toluenesulfonic acid and methanesulfonic acid. Mono-or di-acid salts may be formed, and these salts may exist in hydrated, solvated or substantially anhydrous form. In general, acid addition salts of the compounds of the present disclosure are more soluble in water and various hydrophilic organic solvents and generally exhibit higher melting points compared to free base forms thereof. The selection of suitable salts is known to those skilled in the art. Other non-pharmaceutically acceptable salts, such as oxalate, may be used in the isolation of the compounds of the present disclosure, for example, for laboratory use or subsequent conversion to pharmaceutically acceptable acid addition salts. Examples of inorganic bases that form suitable salts may include lithium, sodium, potassium, calcium, magnesium, or barium hydroxides. Examples of organic bases that form suitable salts may include aliphatic, cycloaliphatic or aromatic organic amines, such as methylamine, trimethylamine and picoline or ammonia. Accordingly, in some examples, salts of the present disclosure to be considered may include alkyl, dialkyl, trialkyl or tetra-alkyl ammonium salts. In a specific embodiment, salts to be considered may include L-arginine, benentamine, benzathine, betaine, calcium hydroxide, choline, theanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(-2-hydroxyethyl)pyrrolidine, sodium, triethanolamine, tromethamine, and zinc salts, but are not limited thereto. In a specific embodiment, salts that may be considered may include Na, Ca, K, Mg, Zn, or other metal salts, but are not limited thereto, and the selection of appropriate salts is known to those skilled in the art.

According to another embodiment of the present disclosure, there is provided a pharmaceutical composition for preventing or treating sarcopenia including the sesquiterpene derivative or a pharmaceutically acceptable salt thereof as an active ingredient.

As used herein, the term “prevention” refers to any action that inhibits or delays the onset of sarcopenia by administering a composition. As used herein, the term “treatment” refers to any action that improves or beneficially changes the symptoms of sarcopenia by administering the composition.

The pharmaceutical composition may inhibit the expression of myostatin mRNA or protein, inhibit the expression of Muscle RING-finger protein-1 (MURF1) mRNA or protein or the expression of forehead box O3 (Foxo3) mRNA or protein, thereby more fundamentally preventing and treating muscle loss and reduced muscle strength.

The pharmaceutical composition includes, for example, a sesquiterpene derivative represented by Chemical Formula 1 or a pharmaceutically acceptable salt thereof as an active ingredient, particularly, desirably the compounds of Chemical Formulas 1-1 to 1-4 or 1-6 to 1-8 above, and more desirably the compound of Chemical Formulas 1-1 or 1-2 above, for the purpose of preventing, treating, or ameliorating sarcopenia through inhibition of the expression of mRNAs or proteins of myostatin, MURF1, and Foxo3.

The pharmaceutical composition provided in the present disclosure may be prepared by methods known in the pharmaceutical field or by methods disclosed in Remington's Pharmaceutical Science (19th ed., 1995), and may be mixed with pharmaceutically acceptable carriers, excipients, diluents, stabilizers, preservatives, etc. to be prepared and used in formulations such as powders, granules, tablets, capsules, or injections. In addition, the composition may be prepared as a sustained-release formulation by including a base used for sustained-release purposes in addition to the active ingredient so that the active ingredient is released slowly.

The pharmaceutically acceptable carrier may further include, for example, a carrier for oral administration or a carrier for parenteral administration. The carrier for oral administration may include lactose, starch, cellulose derivatives, magnesium stearate, stearic acid, and the like. In addition, the carrier for oral administration may contain various drug delivery substances used for oral administration of peptide preparations. In addition, the carrier for parenteral administration may include water, suitable oil, saline, aqueous glucose, glycol, and the like, and further include a stabilizer and a preservative. A suitable stabilizer includes antioxidants such as sodium hydrogen sulfite, sodium sulfite or ascorbic acid. A suitable preservative includes benzalkonium chloride, methyl- or propyl-paraben and chlorobutanol. The pharmaceutical composition of the present disclosure may further include a lubricant, a wetting agent, a sweetening agent, a flavoring agent, an emulsifying agent, a suspending agent, and the like, in addition to the ingredients.

The diluents may include non-aqueous solvents such as propylene glycol, polyethylene glycol, vegetable oils such as olive oil and peanut oil, aqueous solvent such as brine (desirably 0.8% brine), and water containing a buffering medium (desirably 0.05 M phosphate buffer solution), or the like, but are not limited thereto.

The excipients may include starch, glucose, lactose, sucrose, gelatin, malt, rice, wheat flour, chalk, silica gel, sodium stearate, glycerol monostearate, talc, sodium chloride, anhydrous skim milk, glycerol, propylene, glycol, water, ethanol, etc., but are not limited thereto.

The stabilizers may include carbohydrates such as sorbitol, mannitol, starch, sucrose, dextran, glutamate, and glucose, and proteins such as animal such as powdered milk, serum albumin, and casein, vegetable, or microbial proteins, but are not limited thereto.

The preservatives may include thimerosal, merthiolate, gentamicin, neomycin, nystatin, amphotericin B, tetracycline, penicillin, streptomycin, and polymyxin B, but are not limited thereto.

The pharmaceutical composition of the present disclosure may be administered to mammals including humans even by any method, for example, orally or parenterally. The parenteral administration methods may include intravenous, intramuscular, intraarterial, intramedullary, intrathecal, intracardiac, transdermal, subcutaneous, intraperitoneal, intranasal, enteral, topical, sublingual, or rectal administration, but are not limited thereto.

The pharmaceutical composition of the present disclosure may be formulated as formulations for oral administration or parenteral administration according to the routes of administration as described above.

The total effective dose of the pharmaceutical composition of the present disclosure may be administered to patients in a single dose, and may be administered to patients by a fractionated therapy that is administered for a long period of time in a multiple dose. In the pharmaceutical composition of the present disclosure, the content of the active ingredients may vary depending on the severity of disease. The content of the active ingredients may be determined by considering various factors, such as a formulation method, an administration route, and the number of treatments, as well as the age, weight, and health condition of a patient, disease symptoms, and administration time and method. Considering this aspect, those skilled in the art will be able to determine an appropriate effective dose of the composition of the present disclosure. The pharmaceutical composition according to the present disclosure is not particularly limited to the formulations, the administration routes, and the administration methods thereof, as long as the effects of the present disclosure are shown.

According to another embodiment of the present disclosure, there is provided a cosmetic composition for preventing or ameliorating sarcopenia including the sesquiterpene derivative or a cosmetically acceptable salt thereof as an active ingredient.

As used herein, the term “ameliorating” means all actions that at least reduce parameters related with conditions to be treated, e.g., the degree of symptoms, or improve and beneficially change the disease.

The cosmetic composition includes, for example, the sesquiterpene derivative represented by Chemical Formula 1 or the cosmetically acceptable salt thereof as an active ingredient, and may be prepared in the form of a basic cosmetic composition (lotion, cream, essence, face cleanser such as cleansing foam and cleansing water, packs, and body oils), a colored cosmetic composition (foundation, lipstick, mascara, and makeup base), a hair product composition (shampoo, conditioner, hair conditioner, and hair gel), soap, etc., in addition to cosmetically acceptable excipients.

The excipients may include, for example, emollients, skin penetration enhancers, colorants, fragrances, emulsifiers, thickeners and solvents, more specifically starch, glucose, lactose, sucrose, gelatin, malt, rice, wheat flour, chalk, silica gel, sodium stearate, glycerol monostearate, talc, sodium chloride, anhydrous skim milk, glycerol, propylene, glycol, water, ethanol, etc., but are not limited thereto.

According to yet another embodiment of the present disclosure, there is provided a food composition for preventing or ameliorating sarcopenia including the sesquiterpene derivative or a food acceptable salt thereof as an active ingredient.

The food composition includes, for example, the sesquiterpene derivative represented by Chemical Formula 1 or a pharmaceutically acceptable salt thereof as an active ingredient, and when the sesquiterpene derivative is used as an additive to the food composition, the sesquiterpene derivative may be added as it is or used with other foods or food ingredients, and may be used appropriately according to conventional methods. In general, when preparing foods or beverages, the composition of the present disclosure is added in an amount of 15 wt % or less, desirably 10 wt % or less, based on the raw material. However, in the case of long-term intake for the purpose of health and hygiene or for the purpose of health control, the amount of the active ingredients may be equal to or greater than the range, and since there is no problem in terms of safety, the active ingredients may be used even in an amount above the range. That is, the mixing amount of the active ingredients may be appropriately determined depending on each purpose of use, such as prevention, health, or treatment.

The formulation of the food composition is able to be in the form of powders, granules, pills, tablets, capsules, as well as general foods or beverages.

The kind of food is not particularly limited. Examples of the food which may be added with the materials include meat, sausages, bread, chocolate, candies, snacks, confectionery, pizza, ramen, other noodles, gums, dairy products including ice cream, various soups, beverages, tea, drinks, alcohol drinks, vitamin complex, and the like, and include all health functional foods in an accepted meaning.

The food of the present disclosure may be prepared by methods which are commonly used in the art and may be prepared by adding raw materials and ingredients which are commonly added in the art in the preparation. Specifically, the food may include proteins, carbohydrates, fats, nutrients, seasonings, and flavoring agents, and examples of the carbohydrates include glucose, fructose, maltose, sucrose, oligosaccharides, dextrin, cyclodextrin, xylitol, sorbitol, erythrocyte, saccharin, or synthetic flavoring agents, but are not limited thereto.

According to yet another embodiment of the present disclosure, there is provided a feed composition for preventing or ameliorating sarcopenia including the sesquiterpene derivative or a feed acceptable salt thereof as an active ingredient.

The feed composition includes, for example, the sesquiterpene derivative represented by Chemical Formula 1 or a feed acceptable salt thereof as an active ingredient. As used herein, the term “feed” may mean any natural or artificial diet, one-meal diet, or ingredients of one-meal diet to be eaten and ingested by livestock or suitable thereto. The feed may include feed additives or supplementary feed.

The type of feed is not particularly limited, and may use feeds commonly used in the art. Non-limiting examples of the feed may include vegetable feeds, such as grains, root fruits, food processing by-products, algae, fibers, pharmaceutical by-products, oils and fats, starches, meals or grain by-products; and animal feeds such as proteins, inorganic materials, oils and fats, minerals, oils and fats, single-cell proteins, animal planktons, foods, etc. These feeds may be used alone or in combination of two or more kinds.

### Mode for Carrying Out the Invention

Hereinafter, the present disclosure will be described in more detail with reference to Examples. The following Examples are described for the purpose of illustrating the present disclosure, and the scope of the present disclosure is not limited thereto.

### EXAMPLE 1. PREPARATION METHOD OF NOVEL SESQUITERPENE DERIVATIVE

The present disclosure provides a preparation method of a pharmaceutical composition for preventing or treating sarcopenia including a sesquiterpene derivative represented by Chemical Formula 1 or a pharmaceutically acceptable salt thereof as an active ingredient.

The preparation method of the pharmaceutical composition for preventing or treating sarcopenia including the sesquiterpene derivative represented by Chemical Formula 1 or the pharmaceutically acceptable salt thereof as the active ingredient includes acquiring a compound represented by Chemical Formula 1 below by reacting a compound represented by Chemical Formula 2 and a compound represented by Chemical Formula 3.

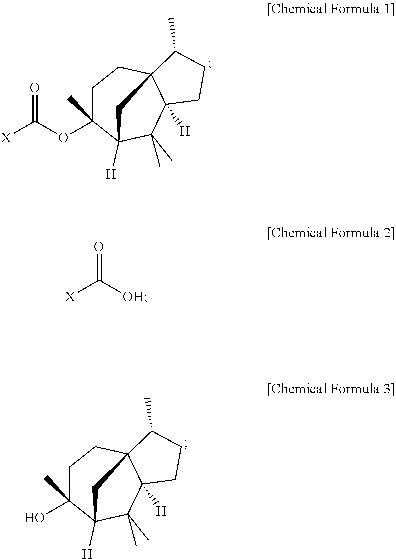

In Chemical Formula, X is

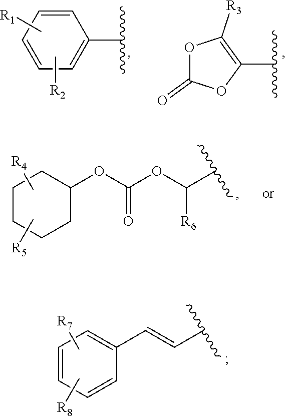

R₁ to R₈ are each independently hydrogen, halogen, straight or branched C1-C5 alkyl, straight or branched C2-C5 alkenyl, C2-C5 alkynyl, C3-C7 cycloalkyl, straight or branched C1-C5 alkoxy, —C(═O)Y₁, —OC(═O)Y₂, cyano, or hydroxyl (wherein one or more hydrogens of the C1-C5 alkyl, C3-C7 cycloalkyl, or C1-C5 alkoxy are each independently unsubstituted or substituted with one or more substituents selected from the group consisting of halogen, hydroxy, cyano, nitro, NY₃Y₄, and

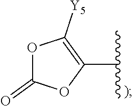

Y₁ to Y₅ are each independently hydrogen, straight or branched C1-C5 alkyl, or C3-C7 cycloalkyl (wherein one or more hydrogens of the C1-C5 alkyl or C3-C7 cycloalkyl are independently unsubstituted or substituted with one or more substituents selected from the group consisting of halogen, hydroxy, cyano, nitro, NZ₁Z₂, and

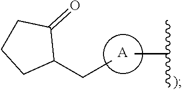

Z₁ and Z₂ are each independently hydrogen or straight or branched C1-C5 alkyl; and A is a C3-C7 aryl group or a C3-C7 heteroaryl group.

### 1.1. Preparation of (3R,3aS,6R,7R,8aS)-3,6,8,8-tetramethyloctahydro-1H-3a,7-metanoazulen-6-yl-4-hydroxy-3-methoxybenzoate [Compound 1-1]

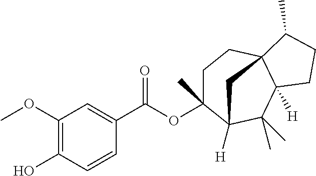 [SMILES: COc1cc(C(=O)O[C@]2(C)CC[C@@]34C[C@@H]2C(C)(C)[C@@H]3CC[C@H]4C)ccc1O]

13.3 g (1.2 equiv.) of (3R,3aS,6R,7R,8aS)-3,6,8,8-tetramethyloctahydro-1H-3a,7-metanoazulen-6-ol and 8.4 g (0.05 mol, 1 equiv.) of 4-hydroxy-3-methoxybenzoate were added in 250 mL of dimethylacetamide and stirred and dissolved. The solution was added sequentially with 103.6 g (1.5 equiv.) of potassium carbonate, 9.5 g (1.2 equiv.) of 4-toluenesulfonyl chloride, and 0.9 g (15 mol %) of 4-dimethylaminopyridine and stirred at room temperature for 2 hours. The mixture was added with a mixed solution of 550 mL of ethyl acetate, 53.6 g of ammonium chloride, and 600 mL of purified water and stirred for 30 minutes. The organic layer was collected, dried with anhydrous magnesium sulfate, filtered, and then concentrated under reduced pressure to obtain 13.4 g (72%) of the labeled compound (3R,3aS,6R,7R,8aS)-3,6,8,8-tetramethyloctahydro-1H-3 a,7-metano azulen-6-yl-4-hydroxy-3-methoxybenzoate.

¹H-NMR(400 MHz, CDCl3) δ0.99 (9H), 1.2-1.3 (4H), 1.46 (2H), 1.48 (3H), 1.56 (1H), 1.66 (3H), 1.72 (1H), 1.95 (2H), 3.83 (3H), 5.35 (1H), 7.15 (1H), 7.45 (1H), 7.46 (1H).

### 1.2. Preparation of (3R,3aS,6R,7R,8aS)-3,6,8,8-tetramethyloctahydro-1H-3a,7-methanoazulen-6-yl-4-acetoxy-3-methoxybenzoate [Compound 1-2]

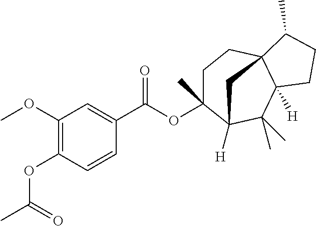 [SMILES: COc1cc(C(=O)O[C@]2(C)CC[C@@]34C[C@@H]2C(C)(C)[C@@H]3CC[C@H]4C)ccc1OC(C)=O]

18.6 g (0.05 mol, 1 equiv.) of (3R,3aS,6R,7R,8aS)-3,6,8,8-tetramethyl octahydro-1H-3a,7-metano azulen-6-yl-4-hydroxy-3-methoxybenzoate prepared in Example 1 was added in 250 mL of dichloromethane and stirred. The solution was added sequentially with 0.9 g (15 mol %) of 4-dimethylaminopyridine, 4.71 g (1.2 equiv.) of acetyl chloride, and 10.5 mL (1.5 equiv.) of triethylamine and stirred at room temperature for 2 hours. When the reaction was completed, 600 mL of purified water was added and stirred for 30 minutes. The stirring was stopped, layers were separated, an organic layer was collected, and an aqueous layer was extracted twice with 500 mL of dichloromethane. The organic layer was washed with water, brine, and a saturated sodium hydrogen carbonate solution, dried using sodium sulfate, and the solvent was concentrated under reduced pressure to obtain a crude compound. The crude compound was recrystallized by adding hexane to obtain 17.6 g (85%) of the labeded compound (3R,3aS,6R,7R,8aS)-3,6,8,8-tetramethyloctahydro-1H-3a,7-methanoazulen-6-yl-4-acetoxy-3-methoxybenzoate.

¹H-NMR(400 MHz, CDCl3) δ0.99 (9H), 1.2-1.3 (4H), 1.46 (2H), 1.48 (3H), 1.56 (1H), 1.66 (3H), 1.72 (1H), 1.95 (2H), 2.28 (3H), 3.83 (3H), 7.29 (1H), 7.59 (1H), 7.62 (1H).

### 1.3. Preparation of (3R,3aS,6R,7R,8aS)-3,6,8,8-tetramethyloctahydro-1H-3a,7-methanoazulen-6-yl-3-methoxy-4-piyaloyloxybenzoate [Compound 1-3]

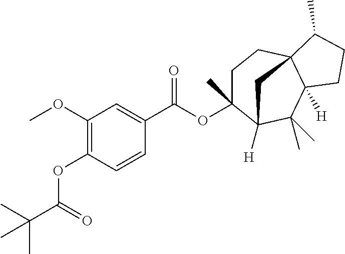 [SMILES: COc1cc(C(=O)O[C@]2(C)CC[C@@]34C[C@@H]2C(C)(C)[C@@H]3CC[C@H]4C)ccc1OC(=O)C(C)(C)C]

17.1 g (0.05 mol, 1 equiv.) of (3R,3aS,6R,7R,8aS)-3,6,8,8-tetramethyloctahydro-1H-3a,7-methanoazulen-6-yl-4-hydroxybenzoate prepared in Example 1 was added in 250 mL of dichloromethane and stirred. The solution was added sequentially with 0.9 g (15 mol %) of 4-dimethylaminopyridine, 7.2 g (1.2 equiv.) of pivaloyl chloride, and 10.5 mL (1.5 equiv.) of triethylamine and stirred at room temperature for 4 hours. When the reaction was completed, 600 mL of cold purified water was added and stirred for 30 minutes. The stirring was stopped, layers were separated, an organic layer was collected, and an aqueous layer was extracted twice with 500 mL of dichloromethane. The organic layer was washed with water, brine, and a saturated sodium hydrogen carbonate solution, dried using sodium sulfate, and the solvent was concentrated under reduced pressure to obtain a crude compound. The crude compound was recrystallized by adding hexane to obtain 16.4 g (72%) of the labeled compound (3R,3aS,6R,7R,8aS)-3,6,8,8-tetramethyloctahydro-1H-3a,7-methanoazulen-6-yl-3-methoxy-4-pivaloyloxybenzoate.

¹H-NMR(400 MHz, CDCl3) δ0.99 (9H), 1.2-1.3 (4H), 1.23 (9H), 1.46 (2H), 1.48 (3H), 1.56 (1H), 1.66 (3H), 1.72 (1H), 1.95 (2H), 3.83 (3H), 7.29 (J=7.5), 7.59 (1H), 7.62 (1H).

### 1.4. Preparation of (3R,3aS,6R,7R,8aS)-3,6,8,8-tetramethyloctahydro-1H-3a,7-methanoazulen-6-yl-5-methyl-2-oxo-1,3-dioxole-4-carboxylate [Compound 1-4]

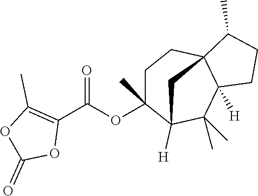 [SMILES: Cc1oc(=O)oc1C(=O)O[C@]1(C)CC[C@@]23C[C@@H]1C(C)(C)[C@@H]2CC[C@H]3C]

13.3 g (1.2 equiv.) of (3R,3aS,6R,7R,8aS)-3,6,8,8-tetramethyloctahydro-1H-3a,7-metanoazulen-6-ol and 7.2 g (0.05 mol, 1 equiv.) of 5-methyl-2-oxo-1,3-dioxole-4-carboxylic acid were added in 250 mL of dimethylacetamide and stirred and dissolved. The solution was added sequentially with 12.4 g (1.2 equiv.) of N,N′-dicyclohexylcarbodiimide and 0.9 g (15 mol %) of 4-dimethylaminopyridine and stirred at room temperature for 5 hours. The mixture was filtered under reduced pressure using a membrane filter (25 μm membrane filter), and the filtrate was added with a mixed solution of 550 mL of ethyl acetate, 53.6 g of ammonium chloride, and 600 mL of purified water and stirred for 30 minutes. The organic layer was collected, dried with anhydrous magnesium sulfate, filtered, and then concentrated under reduced pressure to obtain 9.75 g (56%) of the labeled compound (3R,3aS,6R,7R,8aS)-3,6,8,8-tetramethyl octahydro-1H-3a,7-methanoazulen-6-yl-5 -methyl-2-oxo-1,3-dioxole-4-carboxylate.

¹H-NMR(400 MHz, CDCl3) δ0.99 (9H), 1.2 (1H),1.35 (3H), 1.46 (2H), 1.48 (3H), 1.56 (2H), 1.66 (3H), 1.76 (1H), 1.99 (1H), 2.51 (3H).

### 1.5. Preparation of (3R,3aS,6R,7R,8aS)-3,6,8,8-tetramethyloctahydro-1H-3a,7-methanoazulen-6-yl-2-(((cyclohexyloxy)carbonyDoxy)propanate [Compound 1-5]

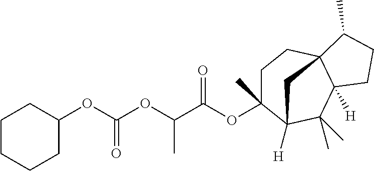 [SMILES: CC(OC(=O)OC1CCCCC1)C(=O)O[C@]1(C)CC[C@@]23C[C@@H]1C(C)(C)[C@@H]2CC[C@H]3C]

13.3 g (1.2 equiv.) of (3R,3aS,6R,7R,8aS)-3,6,8,8-tetramethyloctahydro-1H-3a,7-metanoazulen-6-ol and 10.8 g (0.05 mol, 1 equiv.) of (((cyclohexyloxy)carbonyl)oxy)propionic acid were added in 250 mL of dimethylacetamide and stirred and dissolved. The solution was added sequentially with 12.4 g (1.2 equiv.) of N,N′-dicyclohexylcarbodiimide and 0.9 g (15 mol %) of 4-dimethylaminopyridine and stirred at room temperature for 10 hours. The mixture was filtered under reduced pressure using a membrane filter and the filtrate was added with a mixed solution of 550 mL of ethyl acetate, 53.6 g of ammonium chloride, and 600 mL of purified water and stirred for 30 minutes. The organic layer was collected, dried with anhydrous magnesium sulfate, filtered, and then concentrated under reduced pressure to obtain 12.6 g (60%) of the labeled compound (3R,3aS,6R,7R,8aS)-3,6,8,8-tetramethyloctahydro-1H-3a,7-methanoazulen-6-yl-2-(((cyclohexyloxy)carbonyl)oxy)propanate.

¹H-NMR(400 MHz, CDCl3) δ0.99 (9H), 1.2-1.8 (28H), 1.99 (1H), 3.91 (1H), 4.98 (1H).

### 1.6. Preparation of (3R,3aS,6R,7R,8aS)-3,6,8,8-tetramethyloctahydro-1H-3a,7-methanoazulen-6-yl-4-(pivaloyloxy)benzoate [Compound 1-6]

### 1.6.1. Preparation of (3R,3aS,6R,7R,8aS)-3,6,8,8-tetramethyloctahydro-1H-3a,7-methanoazulen-6-yl-4-hydroxybenzoate

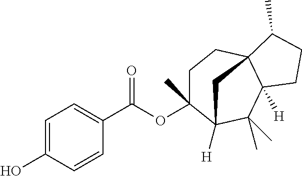 [SMILES: C[C@@H]1CC[C@H]2C(C)(C)[C@H]3C[C@@]12CC[C@@]3(C)OC(=O)c1ccc(O)cc1]

13.3 g (1.2 equiv.) of (3R,3aS,6R,7R,8aS)-3,6,8,8-tetramethyloctahydro-1H-3a,7-metanoazulen-6-ol and 6.9 g (0.05 mol, 1 equiv.) of 4-hydroxybenzoic acid were added in 250 mL of dimethylacetamide and stirred and dissolved. The solution was added sequentially with 103.6 g (1.5 equiv.) of potassium carbonate, 9.5 g (1.2 equiv.) of 4-toluenesulfonyl chloride, and 0.9 g (15 mol %) of 4-dimethylaminopyridine and stirred at room temperature for 2 hours. The mixture was added with a mixed solution of 550 mL of ethyl acetate, 53.6 g of ammonium chloride, and 600 mL of purified water and stirred for 30 minutes. The organic layer was collected, dried with anhydrous magnesium sulfate, filtered, and then concentrated under reduced pressure to obtain 10.8 g (63%) of the labeled compound (3R,3aS,6R,7R,8aS)-3,6,8,8-tetramethyloctahydro-1H-3a,7-methanoazulen-6-yl-4-hydroxybenzoate.

¹H-NMR(400 MHz, CDCl3) δ0.99 (9H), 1.2-1.3 (4H), 1.23 (9H), 1.46 (2H), 1.48 (3H), 1.56 (1H), 1.66 (3H), 1.72 (1H), 1.95 (2H), 5.35 (1H), 6.81 (2H), 7.90 (2H).

### 1.6.2. Preparation of (3R,3aS,6R,7R,8aS)-3,6,8,8-tetramethyloctahydro-1H-3a,7-methanoazulen-6-yl-4-(pivaloyloxy)benzoate

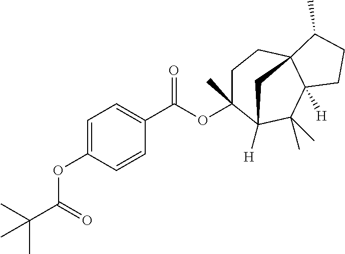 [SMILES: C[C@@H]1CC[C@H]2C(C)(C)[C@H]3C[C@@]12CC[C@@]3(C)OC(=O)c1ccc(OC(=O)C(C)(C)C)cc1]

18.6 g (0.05 mol, 1 equiv.) of (3R,3aS,6R,7R,8aS)-3,6,8,8-tetramethyl octahydro-1H-3a,7-metanoazulen-6-yl-4-hydroxy-3-methoxybenzoate prepared in Example 6-1 was added in 250 mL of dichloromethane and stirred. The solution was added sequentially with 0.9 g (15 mol %) of 4-dimethylaminopyridine, 7.2 g (1.2 equiv.) of pivaloyl chloride, and 10.5 mL (1.5 equiv.) of triethylamine and stirred at room temperature for 4 hours. When the reaction was completed, 600 mL of cold purified water was added and stirred for 30 minutes. The stirring was stopped, layers were separated, an organic layer was collected, and an aqueous layer was extracted twice with 500 mL of dichloromethane. The organic layer was washed with water, brine, and a saturated sodium hydrogen carbonate solution, dried using sodium sulfate, and the solvent was concentrated under reduced pressure to obtain a crude compound. The crude compound was recrystallized by adding hexane to obtain 16.2 g (76%) of the labeled compound (3R,3aS,6R,7R,8aS)-3,6,8,8-tetramethyloctahydro-1H-3a,7-methanoazulen-6-yl-4-(pivaloyloxy)benzoate.

¹H-NMR(400 MHz, CDCl3) δ0.99 (9H), 1.2-1.3 (4H), 1.23 (9H), 1.46 (2H), 1.48 (3H), 1.56 (1H), 1.66 (3H), 1.72 (1H), 1.95 (2H), 7.40 (2H), 8.04 (2H).

### 1.7. Preparation of (E)-(3R,3aS,6R,7R,8aS)-3,6,8,8-tetramethyloctahydro-1H-3a,7-methanoazulen-6-yl-3-(4-hydroxyphenyDacrylate [Compound 1-7]

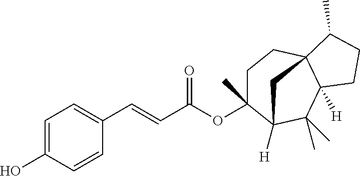 [SMILES: C[C@@H]1CC[C@H]2C(C)(C)[C@H]3C[C@@]12CC[C@@]3(C)OC(=O)/C=C/c1ccc(O)cc1]

16.4 g (0.1 mol, 1.2 equiv.) of (E)-3-(4-hydroxyphenyl)acrylic acid and 26.7 g (1.2 equiv.) of (3R,3aS,6R,7R,8aS)-3,6,8,8-tetramethyloctahydro-1H-3a,7-methanoazulen-6-ol were added to 500 ml of tetrahydrofuran and stirred and dissolved. The solution was added with 4 mL of sulfuric acid, stirred for 30 minutes, and then added with 30 g of magnesium sulfate and the reaction solution was heated and refluxed for 4 hours. When the reaction was completed, the reaction solution was concentrated under reduced pressure to remove tetrahydrofuran, and added with 550 mL of ethyl acetate and 600 mL of a saturated sodium hydrogen carbonate solution and stirred for 30 minutes. The stirring was stopped, layers were separated, an organic layer was collected, and an aqueous layer was extracted twice with 500 mL of ethyl acetate. The organic layer was washed with water and brine, respectively, dried using sodium sulfate, and the solvent was concentrated under reduced pressure to obtain a crude compound. The crude compound was purified by column chromatography (ethyl acetate:hexane=1:5) to obtain 23.6 g (64%) of the labeled compound (E)-(3R,3aS,6R,7R,8aS)-3,6,8,8-tetramethyloctahydro-1H-3a,7-methanoazulen-6-yl-3-(4-hydroxyphenyl)acrylate.

¹H-NMR(400 MHz, CDCl3) δ0.99 (9H), 1.2-1.3 (4H), 1.46 (2H), 1.48 (3H), 1.56 (2H), 1.66 (3H), 1.72 (1H), 1.99 (1H), 5.35 (1H), 6.30 (1H), 6.65 (2H), 7.48 (1H), 7.56 (2H).

### 1.8. Preparation of (E)-(3R,3aS,6R,7R,8aS)-3,6,8,8-tetramethyloctahydro-1H-3a,7-methanoazulen-6-yl-3-(4-hydroxy-3-methoxyphenyDacrylate [Compound 1-8]

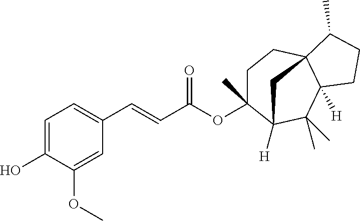 [SMILES: COc1cc(/C=C/C(=O)O[C@]2(C)CC[C@@]34C[C@@H]2C(C)(C)[C@@H]3CC[C@H]4C)ccc1O]

9.7 g (0.05 mol, 1 equiv.) of (E)-3-(4-hydroxy-3-methoxyphenyl)acrylic acid and 13.3 g (1.2 equiv.) of (3R,3aS,6R,7R,8aS)-3,6,8,8-tetramethyloctahydro-1H-3a,7-methanoazulen-6-ol were added to 250 ml of tetrahydrofuran and stirred and dissolved. The solution was added with 2 mL of sulfuric acid, stirred for 30 minutes, and then added with 15 g of magnesium sulfate and the reaction solution was heated and refluxed for 6 hours. When the reaction was completed, the reaction solution was concentrated under reduced pressure to remove tetrahydrofuran, and added with 550 mL of ethyl acetate and 600 mL of a saturated sodium hydrogen carbonate solution and stirred for 30 minutes. The stirring was stopped, layers were separated, an organic layer was collected, and an aqueous layer was extracted twice with 500 mL of ethyl acetate. The organic layer was washed with water and brine, respectively, dried using sodium sulfate, and the solvent was concentrated under reduced pressure to obtain a crude compound. The crude compound was purified by column chromatography (ethyl acetate:hexane=1:10) to obtain 13.4 g (67%) of the labeled compound (E)-(3R,3aS,6R,7R,8aS)-3,6,8,8-tetramethyloctahydro-1H-3a,7-methanoazulen-6-yl-3-(4-hydroxy-3-methoxyphenyl)acrylate.

¹H-NMR(400 MHz, CDCl3) δ0.99 (9H), 1.2-1.3 (4H), 1.46 (2H), 1.48 (3H), 1.56 (2H), 1.66 (3H), 1.72 (1H), 1.99 (1H), 3.82 (3H), 5.35 (1H), 6.30 (1H), 6.79 (1H), 6.99 (1H), 7.16 (1H), 7.48 (d, 1H, J=15.1).

### 1.9. Preparation of (E)-(3R,3aS,6R,7R,8aS)-3,6,8,8-tetramethyloctahydro-1H-3a,7-metanoazulen-6-yl-3-(3,4-dihydroxyphenyDacrylate [Compound 1-9]

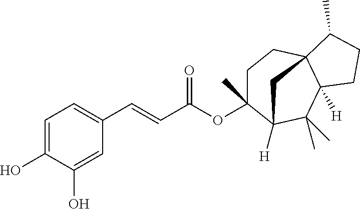 [SMILES: C[C@@H]1CC[C@H]2C(C)(C)[C@H]3C[C@@]12CC[C@@]3(C)OC(=O)/C=C/c1ccc(O)c(O)c1]

9.0 g (0.05 mol) of (E)-3-(3,4-hydroxyphenyl)acrylic acid and 13.3 g (1.2 equiv.) of (3R,3aS,6R,7R,8aS)-3,6,8,8-tetramethyloctahydro-1H-3a,7-methanoazulen-6-ol were added to 250 ml of tetrahydrofuran and stirred and dissolved. The solution was added with 2 mL of sulfuric acid, stirred for 30 minutes, and then added with 15 g of magnesium sulfate and the reaction solution was heated and refluxed for 2.5 hours. When the reaction was completed, the reaction solution was concentrated under reduced pressure to remove tetrahydrofuran, and added with 550 mL of ethyl acetate and 600 mL of a saturated sodium hydrogen carbonate solution and stirred for 30 minutes. The stirring was stopped, layers were separated, an organic layer was collected, and an aqueous layer was extracted twice with 500 mL of ethyl acetate. The organic layer was washed with water and brine, respectively, dried using sodium sulfate, and the solvent was concentrated under reduced pressure to obtain a crude compound. The crude compound was purified by column chromatography (ethyl acetate:hexane=1:3) to obtain 13.8 g (72%) of the labeled compound (E)-(3R,3aS,6R,7R,8aS)-3,6,8,8-tetramethyloctahydro-1H-3a,7-metanoazulen-6-yl-3-(3,4-dihydroxyphenyl)acrylate.

¹H-NMR(400 MHz, CDCl3) δ0.99 (9H), 1.2-1.3 (4H), 1.46 (2H), 1.48 (3H), 1.56 (2H), 1.66 (3H), 1.72 (1H), 1.99 (1H), 5.35 (2H), 6.31 (1H), 6.79 (1H), 6.92 (1H), 7.17 (1H), 7.48 (1H).

### 1.10. Preparation of (E)-(3R,3aS,6R,7R,8aS)-3,6,8,8-tetramethyloctahydro-1H-3a,7-methanoazulen-6-yl cinnamate [Compound 1-10]

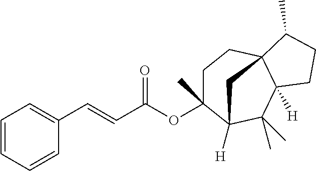 [SMILES: C[C@@H]1CC[C@H]2C(C)(C)[C@H]3C[C@@]12CC[C@@]3(C)OC(=O)/C=C/c1ccccc1]

7.4 g (0.05 mol, 1 equiv.) of cinnamate and 13.3 g (1.2 equiv.) of (3R,3aS,6R,7R,8aS)-3,6,8,8-tetramethyloctahydro-1H-3a,7-methanoazulen-6-ol were added to 250 ml of tetrahydrofuran and added with 0.4 g (5 mol %) of iron chloride and stirred. The reaction solution was heated and refluxed and reacted for 24 hours. After the reaction was completed, the reaction solution was concentrated under reduced pressure to remove the solvent, and then added with 500 mL of ethyl acetate and 600 mL of purified water, and the layers were separated. The organic layer was collected, washed with water and brine, dried with sodium sulfate, and concentrated under reduced pressure to obtain a crude compound. The crude compound was purified by column chromatography (ethyl acetate:hexane=1:10) to obtain 13.1 g (74%) of the labeled compound (E)-(3R,3aS,6R,7R,8aS)-3,6,8,8-tetramethyloctahydro-1H-3a,7-methanoazulen-6-yl cinnamate.

¹H-NMR(400 MHz, CDCl3) δ0.99 (9H), 1.2-1.3 (4H), 1.46 (2H), 1.48 (3H), 1.56 (2H), 1.66 (3H), 1.72 (1H), 1.99 (1H), 6.31 (1H), 7.33-7.40 (3H), 7.48 (1H) 7.60 (2H).

### 1.11. Preparation of (E)-(3R,3aS,6R,7R,8aS)-3,6,8,8-tetramethyloctahydro-1H-3a,7-metanoazulen-6-yl-3-(4-(2-hydroxyethoxy)phenypacrylate [Compound 1-11]

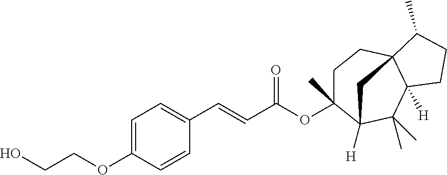 [SMILES: C[C@@H]1CC[C@H]2C(C)(C)[C@H]3C[C@@]12CC[C@@]3(C)OC(=O)/C=C/c1ccc(OCCO)cc1]

18.4 g (0.05 mol, 1 equiv.) of (E)-(3R,3aS,6R,7R,8aS)-3,6,8,8-tetramethyloctahydro-1H-3a,7-methanoazulen-6-yl-3-(4-hydroxy phenyl)acrylate prepared in Example 7 was added in 250 mL of dichloromethane and stirred. The solution was added sequentially with 0.9 g (15 mol %) of 4-dimethylaminopyridine, 4.8 g (1.2 equiv.) of 2-chloroethanol, and 10.5 mL (1.5 equiv.) of triethylamine and stirred at room temperature for 2 hours. When the reaction was completed, 600 mL of cold purified water was added and stirred for 30 minutes. The stirring was stopped, layers were separated, an organic layer was collected, and an aqueous layer was extracted twice with 500 mL of dichloromethane. The organic layer was washed with water, brine, and a saturated sodium hydrogen carbonate solution, dried using sodium sulfate, and the solvent was concentrated under reduced pressure to obtain a crude compound. The crude compound was recrystallized by adding hexane to obtain 13.6 g (66%) of the labeled compound (E)-(3R,3aS,6R,7R,8aS)-3,6,8,8-tetramethyloctahydro-1H-3a,7-methanoazulen-6-yl-3-(4-(2-hydroxyethoxy)phenyl)acrylate.

¹H-NMR(400 MHz, CDCl3) δ0.99 (9H), 1.2-1.3 (4H), 1.46 (2H), 1.48 (3H), 1.56 (2H), 1.66 (3H), 1.72 (1H), 1.99 (1H), 3.67 (3H), 4.33 (2H), 6.31 (1H), 6.94 (2H), 7.48 (1H) 7.60 (2H).

### 1.12. Preparation of (E)-(3R,3aS,6R,7R,8aS)-3,6,8,8-tetramethyloctahydro-1H-3a,7-methanoazulen-6-yl-3-(4-(2-(dimethylamino)ethoxy)phenyl)acrylate [Compound 1-12]

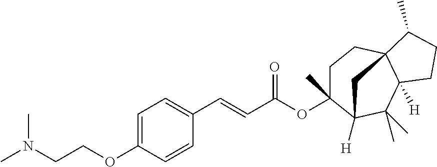 [SMILES: C[C@@H]1CC[C@H]2C(C)(C)[C@H]3C[C@@]12CC[C@@]3(C)OC(=O)/C=C/c1ccc(OCCN(C)C)cc1]

18.4 g (0.05 mol, 1 equiv.) of (E)-(3R,3aS,6R,7R,8aS)-3,6,8,8-tetramethyloctahydro-1H-3a,7-methanoazulen-6-yl-3-(4-hydroxy phenyl)acrylate prepared in Example 7 was added in 250 mL of dichloromethane and stirred. The solution was added sequentially with 0.9 g (15 mol %) of 4-dimethylaminopyridine, 6.5 g (1.2 equiv.) of 2-chloro-N,N-dimethylethenamine, and 10.5 mL (1.5 equiv.) of triethylamine and stirred at room temperature for 3 hours. When the reaction was completed, 600 mL of cold purified water was added and stirred for 30 minutes. The stirring was stopped, layers were separated, an organic layer was collected, and an aqueous layer was extracted twice with 500 mL of dichloromethane. The organic layer was washed with water, brine, and a saturated sodium hydrogen carbonate solution, respectively, dried using sodium sulfate, and the solvent was concentrated under reduced pressure to obtain a crude compound. The crude compound was recrystallized by adding hexane to obtain 16.7 g (76%) of the labeled compound (E)-(3R,3aS,6R,7R,8aS)-3,6,8,8-tetramethyloctahydro-1H-3a,7-methanoazulen-6-yl-3-(4-(2-(dimethylamino)ethoxy)phenyl)acrylate.

¹H-NMR (400 MHz, CDCl3) δ0.99 (m 9H), 1.2-1.3 (4H), 1.46 (2H), 1.48 (3H), 1.56 (2H), 1.66 (3H), 1.72 (1H), 1.99 (1H), 2.76 (2H), 2.82 (6H) 4.11 (2H), 6.31 (1H), 6.94 (2H), 7.48 (1H) 7.62 (2H).

### 1.13. Preparation of (3R,3aS,6R,7R,8aS)-3,6,8,8-tetramethyloctahydro-1H-3a,7-methanoazulen-6-yl benzoate [Compound 1-13]

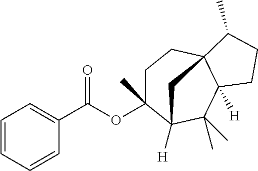 [SMILES: C[C@@H]1CC[C@H]2C(C)(C)[C@H]3C[C@@]12CC[C@@]3(C)OC(=O)c1ccccc1]

6.3 g (2.0 eq) of (3R,3aS,6R,7R,8aS)-3,6,8,8-tetramethyloctahydro-1H-3a,7-metanoazulen-6-ol was dissolved in 100 mL of methylene chloride and then added with 3.4 g (3 eq) of pyridine and stirred at room temperature for 10 minutes. 2 g (0.014 mol, 1 eq) of benzoyl chloride was dissolved in 20 mL of MC and then added dropwise for 1 hour while maintaining the internal temperature of the reaction solution at 20 to 25° C. The reaction solution was heated and stirred under reflux for 4 hours, then added with 80 mL of purified water and 50 mL of brine and stirred for 30 minutes. The organic layer was separated and washed once with 150 mL of a 20% ammonium chloride aqueous solution. The organic layer was separated, washed with 150 mL of purified water, dried with anhydrous sodium sulfate, and then concentrated under reduced pressure. The concentrated residue was added with 50 mL of normal-hexane, stirred for 1 hour, and then filtered to obtain a crude product of (3R,3aS,6R,7R,8aS)-3,6,8,8-tetramethyloctahydro-1H-3a,7-methanoazulen-6-yl benzoate. The obtained crude product was purified by silica gel column chromatography (ethyl acetate:normal-hexane=1:8) to obtain 2.5 g (54%) of the labeled compound (3R,3aS,6R,7R,8aS)-3,6,8,8-tetramethyloctahydro-1H-3a,7-methanoazulen-6-yl benzoate.

¹H-NMR(400 MHz, DMSO-d6) δ0.87-0.90 (9H), 1.00-1.30 (3H) 1.40-1.55 (6H), 1.67-1.69 (3H) 1.70-2.00 (4H), 7.52-7.56 (4H), 7.70 (1H).

### 1.14. Preparation of (3R,3aS,6R,7R,8aS)-3,6,8,8-tetramethyloctahydro-1H-3a,7-methanoazulen-6-yl-4-hydroxybenzoate [Compound 1-14]

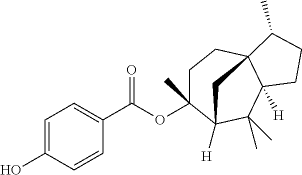 [SMILES: C[C@@H]1CC[C@H]2C(C)(C)[C@H]3C[C@@]12CC[C@@]3(C)OC(=O)c1ccc(O)cc1]

13.3 g (1.2 eq) of (3R,3aS,6R,7R,8aS)-3,6,8,8-tetramethyloctahydro-1H-3a,7-metanoazulen-6-ol and 6.9 g (0.05 mol, 1 eq) of 4-hydroxybenzoic acid were added in 250 mL of dimethylacetamide and stirred and dissolved. The solution was added sequentially with 103.6 g (1.5 eq.) of potassium carbonate, 9.5 g (1.2 eq.) of 4-toluenesulfonyl chloride, and 0.9 g (15 mol %) of 4-dimethylaminopyridine and stirred at room temperature for 2 hours. The mixture was added with a mixed solution of 550 mL of ethyl acetate, 53.6 g of ammonium chloride, and 600 mL of purified water and stirred for 30 minutes. The organic layer was collected, dried with anhydrous magnesium sulfate, filtered, and then concentrated under reduced pressure to obtain 10.8 g (63%) of the labeled compound (3R,3aS,6R,7R,8aS)-3,6,8,8-tetramethyloctahydro-1H-3a,7-methanoazulen-6-yl-4-hydroxybenzoate.

¹H-NMR(400 MHz, CDCl3) δ0.99 (9H), 1.2-1.3 (4H), 1.23 (9H), 1.46 (2H), 1.48 (3H), 1.56 (1H), 1.66 (3H), 1.72 (1H), 1.95 (2H), 5.35 (1H), 6.81 (2H), 7.90 (2H5).

### 1.15. Preparation of (3R,3aS,6R,7R,8aS)-3,6,8,8-tetramethyloctahydro-1H-3a,7-methanoazulen-6-yl4-acetoxybenzoate [Compound 1-15]

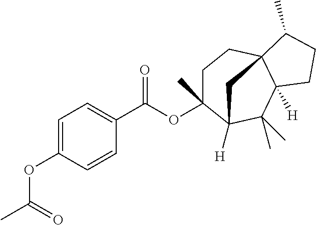 [SMILES: CC(=O)Oc1ccc(C(=O)O[C@]2(C)CC[C@@]34C[C@@H]2C(C)(C)[C@@H]3CC[C@H]4C)cc1]

10.0 g (0.03 mol, 1 eq) of (3R,3aS,6R,7R,8aS)-3,6,8,8-tetramethyloctahydro-1H-3a,7-methanoazulen-6-yl4-hydroxybenzoate prepared in Example 14 was added in 250 mL of dichloromethane and stirred. The solution was added sequentially with 0.5 g (15 mol %) of 4-dimethylaminopyridine, 2.7g (1.2 eq) of acetyl chloride, and 4.4 g (1.5 eq) of triethylamine and stirred at room temperature for 4 hours. The reaction solution was added with 300 mL of purified water and stirred for 30 minutes. An organic layer was separated and then an upper aqueous layer was extracted twice with 200 mL of dichloromethane. The organic layer was collected and washed once with 200 mL of purified water, and then washed sequentially with 200 mL of brine and 200 mL of a saturated sodium bicarbonate aqueous solution. The organic layer was dried with anhydrous sodium sulfate and the solvent was concentrated under reduced pressure to obtain a crude product of (3R,3aS,6R,7R,8aS)-3,6,8,8-tetramethyloctahydro-1H-3a,7-metanoazulen-6-yl 4-acetoxybenzoate. The obtained crude product was purified by silica gel column chromatography (ethyl acetate:normal-hexane=1:10) to obtain 9.5 g (85%) of the labeled compound (3R,3aS,6R,7R,8aS)-3,6,8,8-tetramethyloctahydro-1H-3a,7-methanoazulen-6-yl4-acetoxybenzoate.

¹H-NMR(400 MHz, DMSO-d6) δ0.87-0.90 (9H), 1.01-1.32 (3H), 1.39-1.56 (6H), 1.63-1.65 (3H), 1.75-2.01 (4H), 2.31 (3H), 7.31 (2H), 8.11 (2H).

### 1.16. Preparation of (3R,3aS,6R,7R,8aS)-3,6,8,8-tetramethyloctahydro-1H-3a,7-methanoazulen-6-yl4-(2-hydroxyethoxy)benzoate [Compound 1-16]

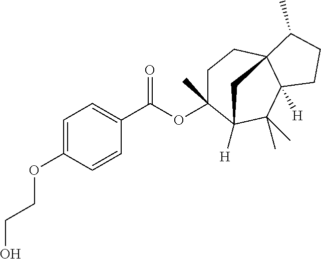 [SMILES: C[C@@H]1CC[C@H]2C(C)(C)[C@H]3C[C@@]12CC[C@@]3(C)OC(=O)c1ccc(OCCO)cc1]

10.0 g (0.03 mol, 1 eq) of (3R,3aS,6R,7R,8aS)-3,6,8,8-tetramethyloctahydro-1H-3a,7-methanoazulen-6-yl4-hydroxybenzoate prepared in Example 14 was added in 250 mL of dichloromethane and stirred. The solution was added sequentially with 0.5 g (15 mol %) of 4-dimethylaminopyridine, 2.8 g (1.2 eq) of 2-chloroethane-1-ol, and 4.4 g (1.5 eq) of triethylamine and stirred at room temperature for 6 hours. The reaction solution was added with 300 mL of purified water and stirred for 30 minutes. An organic layer was separated and then an upper aqueous layer was extracted twice with 200 mL of dichloromethane. The organic layer was collected and washed once with 200 mL of purified water, and then washed sequentially with 200 mL of brine and 200 mL of a saturated sodium bicarbonate aqueous solution. The organic layer was dried with anhydrous sodium sulfate and the solvent was concentrated under reduced pressure to obtain a crude product of (3R,3aS,6R,7R,8aS)-3,6,8,8-tetramethyloctahydro-1H-3a,7-methanoazulen-6-yl 4-(2-hydroxyethoxy)benzoate. The obtained crude product was purified by silica gel column chromatography (ethyl acetate:normal-hexane=1:7) to obtain 8.2 g (73%) of the labeled compound (3R,3aS,6R,7R,8aS)-3,6,8,8-tetramethyl octahydro-1H-3 a,7-methano azul en-6-yl4-(2 -hydroxyethoxy)benzoate.

¹H-NMR(400 MHz, DMSO-d6) δ0.87-0.90 (9H), 1.01-1.33 (3H), 1.39-1.55 (6H), 1.61-1.63 (3H), 1.74-2.02 (4H), 3.67-3.72 (2H), 4.30-4.33 (2H), 4.90 (1H), 7.01 (2H), 7.96 (2H).

### 1.17. Preparation of (3R,3aS,6R,7R,8aS)-3,6,8,8-tetramethyloctahydro-1H-3a,7-methanoazulen-6-yl4-(2-dimethylamino)ethoxy)benzoate [Compound 1-17]

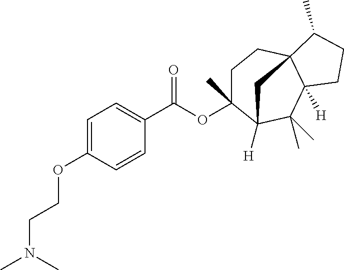 [SMILES: C[C@@H]1CC[C@H]2C(C)(C)[C@H]3C[C@@]12CC[C@@]3(C)OC(=O)c1ccc(OCCN(C)C)cc1]

10.3 g (0.025 mol, 1 equiv.) of (3R,3aS,6R,7R,8aS)-3,6,8,8-tetramethyloctahydro-1H-3a,7-methanoazulen-6-yl 4-hydroxybenzoate prepared in Example 14 was added in 250 mL of dichloromethane and stirred. The solution was added sequentially with 0.45 g (15 mol %) of 4-dimethylaminopyridine, 4.0 g (1.1 equiv.) of 2-chloro-N,N-dimethylethanamine hydrochloride, and 8.7 mL (2.5 equiv.) of triethylamine and stirred at room temperature for 4 hours. When the reaction was completed, 150 mL of cold purified water was added and stirred for 30 minutes. The stirring was stopped, layers were separated, an organic layer was collected, and an aqueous layer was extracted twice with 150 mL of dichloromethane. The organic layer was washed with water and brine, respectively, dried using sodium sulfate, and the solvent was concentrated under reduced pressure to obtain a crude compound. The crude compound was recrystallized by adding hexane to obtain 7.3 g (71%) of the labeled compound (3R,3aS,6R,7R,8aS)-3,6,8,8-tetramethyloctahydro-1H-3a,7-methanoazulen-6-yl 4-(2-dimethylamino)ethoxy)-benzoate.

¹H-NMR(400 MHz, CDCl3) δ0.96(9H), 1.31-2.00 (16H), 2.76 (2H), 2.82 (6H), 4.11 (2H), 6.82 (2H), 7.10 (2H)

### 1.18. Preparation of (3R,3aS,6R,7R,8aS)-3,6,8,8-tetramethyloctahydro-1H-3a,7-methanoazulen-6-yl4-((cyclopentanecarbonyDoxy)benzoate [Compound 1-18]

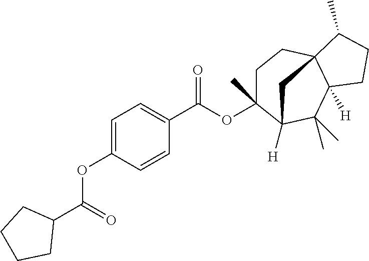 [SMILES: C[C@@H]1CC[C@H]2C(C)(C)[C@H]3C[C@@]12CC[C@@]3(C)OC(=O)c1ccc(OC(=O)C2CCCC2)cc1]

10.3 g (0.025 mol, 1 equiv.) of (3R,3aS,6R,7R,8aS)-3,6,8,8-tetramethyloctahydro-1H-3a,7-methanoazulen-6-yl 4-hydroxybenzoate prepared in Example 14 was added in 250 mL of dichloromethane and stirred. The solution was added sequentially with 6.6 g (2.0 equiv.) of cyclopentanecarbonyl chloride and 6.0 mL (3.0 equiv.) of pyridine and stirred under reflux for 4 hours. When the reaction was completed, 200 mL of cold purified water was added and stirred for 30 minutes. The stirring was stopped, layers were separated, an organic layer was collected, and an aqueous layer was extracted twice with 200 mL of dichloromethane. The organic layer was washed with 300 mL of an ammonium chloride aqueous solution and 300 mL of water, dried with sodium sulfate, and concentrated under reduced pressure to obtain a crude compound. The crude compound was recrystallized by adding hexane to obtain 6.8 g (62%) of the labeled compound (3R,3aS,6R,7R,8aS)-3,6,8,8-tetramethyloctahydro-1H-3a,7-metanoazulen-6-yl 4-((cyclopentanecarbonyl)oxy)benzoate.

¹H-NMR(400 MHz, CDCl3) δ0.99 (9H), 1.30-1.99 (24H), 2.32 (1H), 7.42 (2H), 8.01 (2H)

### 1.19. Preparation of (3R,3aS,6R,7R,8aS)-3,6,8,8-tetramethyloctahydro-1H-3a,7-methanoazulen-6-yl4-((cyclohexanecarbonyDoxy)benzoate [Compound 1-19]

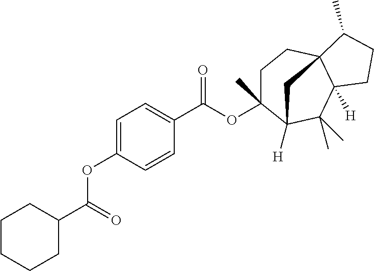 [SMILES: C[C@@H]1CC[C@H]2C(C)(C)[C@H]3C[C@@]12CC[C@@]3(C)OC(=O)c1ccc(OC(=O)C2CCCCC2)cc1]

3.4 g (0.01 mol, 1 equiv.) of (3R,3aS,6R,7R,8aS)-3,6,8,8-tetramethyloctahydro-1H-3a,7-methanoazulen-6-yl 4-hydroxybenzoate prepared in Example 14 was added in 250 mL of dichloromethane and stirred. The solution was added sequentially with 2.9 g (2.0 equiv.) of cyclopentanecarbonyl chloride and 2.4 mL (3.0 equiv.) of pyridine and stirred under reflux for 3 hours. When the reaction was completed, 200 mL of cold purified water was added and stirred for 30 minutes. The stirring was stopped, layers were separated, an organic layer was collected, and an aqueous layer was extracted twice with 200 mL of dichloromethane. The organic layer was washed with 300 mL of an ammonium chloride aqueous solution and 300 mL of water, dried with sodium sulfate, and concentrated under reduced pressure to obtain a crude compound. The crude compound was recrystallized by adding hexane to obtain 3.5 g (77%) of the compound (3R,3aS,6R,7R,8aS)-3,6,8,8-tetramethyloctahydro-1H-3a,7-methanoazulen-6-yl 4-((cyclohexanecarbonyl)oxy)benzoate.

¹H-NMR(400 MHz, CDCl3) δ0.96-1.00 (15H), 1.31-2.00 (18H), 3.52 (1H), 5.14 (2H), 7.42 (2H), 8.02 (2H)

### 1.20. Preparation of (3R,3aS,6R,7R,8aS)-3,6,8,8-tetramethyloctahydro-1H-3a,7-methanoazulen-6-yl 4-(((S)-2-amino-4-methylpentanoyl)oxy)benzoate [Compound 1-20]

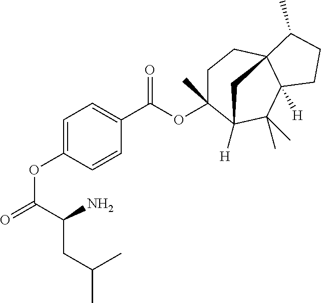 [SMILES: CC(C)C[C@H](N)C(=O)Oc1ccc(C(=O)O[C@]2(C)CC[C@@]34C[C@@H]2C(C)(C)[C@@H]3CC[C@H]4C)cc1]

6.8 g (0.02 mol, 1 equiv.) of (3R,3aS,6R,7R,8aS)-3,6,8,8-tetramethyloctahydro-1H-3a,7-methanoazulen-6-yl 4-hydroxybenzoate prepared in Example 14 was added in 250 mL of dichloromethane and stirred. The solution was added sequentially with 3.1 g (1.2 equiv.) of L-leucine, 5.7 g (1.5 equiv.) of p-toluenesulfonyl chloride, and 5.5 g (2.0 equiv.) of potassium carbonate and stirred at 50° C. for 4 hours. When the reaction was completed, 200 mL of cold purified water and 200 mL of ethyl acetate were added and stirred for 30 minutes. The stirring was stopped, layers were separated, an organic layer was collected, and an aqueous layer was extracted twice with 200 mL of ethyl acetate. The organic layer was washed with 300 mL of brine and 300 mL of water, dried with sodium sulfate, and concentrated under reduced pressure to obtain a crude compound. The crude compound was purified by column chromatography (ethyl acetate:hexane =1:5) to obtain 5.1 g (56%) of the labeled compound (3R,3aS,6R,7R,8aS)-3,6,8,8-tetramethyloctahydro-1H-3a,7-methanoazulen-6-yl 4-(((S)-2-amino- 4-methylpentanoyl)oxy)benzoate.

¹H-NMR(400 MHz, CDCl3) δ0.96 (9H), 1.31-2.00 (26H), 2.32 (1H), 7.42 (2H), 8.02 (2H).

### 1.21. Preparation of (E)-(3R,3aS,6R,7R,8aS)-3,6,8,8-tetramethyloctahydro-1H-3a,7-methanoazulen-6-yl-3-(4-methoxy) cinnamate [Compound 1-21]

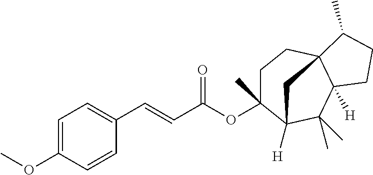 [SMILES: COc1ccc(/C=C/C(=O)O[C@]2(C)CC[C@@]34C[C@@H]2C(C)(C)[C@@H]3CC[C@H]4C)cc1]

40 ml of tetrahydrofuran was added to 3.0 g (16.8 mmol, 1 equiv.) of 4-methoxy cinnamic acid and stirred and dissolved. The solution was added sequentially with 2.7 mL of N,N-dimethylformamide and 4.0 g (33.7 mmol, 3 equiv.) of thionyl chloride and stirred at room temperature for 2 hours. The solvent was concentrated under reduced pressure, added with 20 mL of dichloromethane and subjected to azeotropic distillation.

In another reactor, 7.5 g (2.0 equiv.) of (3R,3aS,6R,7R,8aS)-3,6,8,8-tetramethyloctahydro-1H-3a,7-methanoazulen-6-ol was added in 100 mL of dichloromethane and stirred and dissolved. The solution was added with 4.1 mL (3.0 equiv.) of pyridine and oil separately concentrated under reduced pressure was dissolved in 20 mL of dichloromethane, sequentially added, and stirred under reflux for 6 hours. After the reaction was completed, 100 mL of brine was added and the layers were separated. An organic layer was washed with 100 mL of an ammonium chloride aqueous solution and 100 mL of water, dried with sodium sulfate, and concentrated under reduced pressure to obtain a crude compound. The crude compound was purified by column chromatography (ethyl acetate:hexane=1:10) to obtain 4.6 g (72%) of the labeled compound (E)-(3R,3aS,6R,7R,8aS)-3,6,8,8-tetramethylo ctahydro-1H-3a,7-methanoazulen-6-yl-3-(4-methoxy) cinnamate.

¹H-NMR(400 MHz, CDCl3) δ0.90 (9H), 1.22-1.32 (6H), 1.62 (5H), 1.84 (2H), 1.88 (2H), 2.52 (1H), 3.83 (3H), 6.21-6.25 (1H), 6.88-6.90 (2H), 7.45 (2H) 7.47-7.55 (1H)

### 1.22. Preparation of (E)-(3R,3aS,6R,7R,8aS)-3,6,8,8-tetramethyloctahydro-1H-3a,7-methanoazulen-6-yl-3-(4-(pivaloyloxy)phenypacrylate [Compound 1-22]

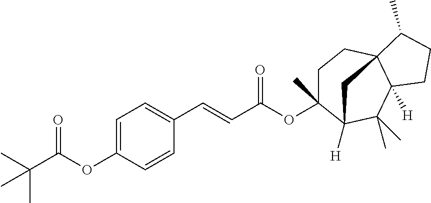 [SMILES: C[C@@H]1CC[C@H]2C(C)(C)[C@H]3C[C@@]12CC[C@@]3(C)OC(=O)/C=C/c1ccc(OC(=O)C(C)(C)C)cc1]

18.4 g (0.05 mol, 1 equiv.) of (E)-(3R,3aS,6R,7R,8aS)-3,6,8,8-tetramethyloctahydro-1H-3a,7-methanoazulen-6-yl-3-(4-hydroxy phenyl)acrylate prepared in Example 7 was added in 250 mL of dichloromethane and stirred. The solution was added sequentially with 0.9 g (15 mol %) of 4-dimethylaminopyridine, 7.2 g (1.2 equiv.) of pivaloyl chloride, and 10.5 mL (1.5 equiv.) of triethylamine and stirred at room temperature for 3 hours. When the reaction was completed, 600 mL of cold purified water was added and stirred for 30 minutes. The stirring was stopped, layers were separated, an organic layer was collected, and an aqueous layer was extracted twice with 500 mL of dichloromethane. The organic layer was washed with water, brine, and a saturated sodium hydrogen carbonate solution, respectively, dried using sodium sulfate, and the solvent was concentrated under reduced pressure to obtain a crude compound. The crude compound was recrystallized by adding hexane to obtain 17.0 g (75%) of the labeled compound (E)-(3R,3aS,6R,7R,8aS)-3,6,8,8-tetramethyloctahydro-1H-3a,7-methanoazulen-6-yl-3-(4-(2 (pivaloyloxy)phenyl)acrylate.

¹H-NMR (400 MHz, CDCl3) δ0.89 (9H), 1.01 (1H), 1.15 (1H), 1.31 (10H), 1.41 (5H), 1.56 (2H), 1.65 (3H), 1.90 (3H), 6,3 (1H), 7.3 (2H), 7.5 (1H), 7.6 (2H),

### 1.23. Preparation of (E)-(3R,3aS,6R,7R,8aS)-3,6,8,8-tetramethyloctahydro-1H-3a,7-methanoazulen-6-yl-3-(4-medoxomil oxy)phenyl)acrylate [Compound 1-23]

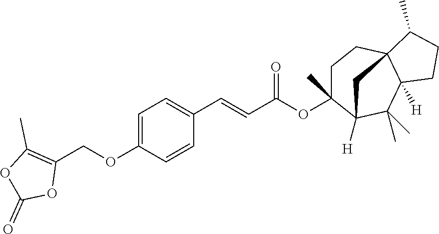 [SMILES: Cc1oc(=O)oc1COc1ccc(/C=C/C(=O)O[C@]2(C)CC[C@@]34C[C@@H]2C(C)(C)[C@@H]3CC[C@H]4C)cc1]

18.4 g (0.05 mol, 1 equiv.) of (E)-(3R,3aS,6R,7R,8aS)-3,6,8,8-tetramethyloctahydro-1H-3a,7-methanoazulen-6-yl-3-(4-hydroxyphenyl)acrylate prepared in Example 7 was added in 250 mL of dichloromethane and stirred. The solution was added sequentially with 0.9 g (15 mol %) of 4-dimethylaminopyridine, 8.9 g (1.2 equiv.) of medoxomil chloride, and 10.5 mL (1.5 equiv.) of triethylamine and stirred at room temperature for 3 hours. When the reaction was completed, 600 mL of cold purified water was added and stirred for 30 minutes. The stirring was stopped, layers were separated, an organic layer was collected, and an aqueous layer was extracted twice with 500 mL of dichloromethane. The organic layer was washed with water, brine, and a saturated sodium hydrogen carbonate solution, respectively, dried using sodium sulfate, and the solvent was concentrated under reduced pressure to obtain a crude compound. The crude compound was recrystallized by adding hexane to obtain 16.8 g (70%) of the labeled compound E)-(3R,3aS,6R,7R,8aS)-3,6,8,8-tetramethyloctahydro-1H-3a,7-methanoazulen-6-yl-3-(4-(medoxomil oxy)phenyl)acrylate.

¹H-NMR (400 MHz, CDCl3) δ0.89 (9H), 1.01 (1H), 1.15 (1H), 1.31 (1H), 1.41 (5H), 1.56 (2H), 1.65 (3H), 1.90 (3H), 2.26 (3H), 4.61 (2H), 6,3 (1H), 7.3 (2H), 7.5 (1H), 7.6 (2H).

### 1.24. Preparation of (E)-(3R,3aS,6R,7R,8aS)-3,6,8,8-tetramethyloctahydro-1H-3a,7-methanoazulen-6-yl-3-(3-methoxy-4-pivaloyloxy)phenyl)acrylate [Compound 1-24]

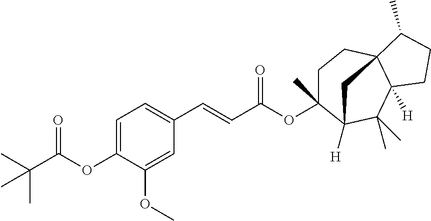 [SMILES: COc1cc(/C=C/C(=O)O[C@]2(C)CC[C@@]34C[C@@H]2C(C)(C)[C@@H]3CC[C@H]4C)ccc1OC(=O)C(C)(C)C]

19.9 g (0.05 mol, 1 equiv.) of (E)-(3R,3aS,6R,7R,8aS)-3,6,8,8-tetramethyloctahydro-1H-3a,7-metanoazulen-6-yl-3-(4-hydroxy-3-methoxyphenyl)acrylate prepared in Example 8 was added in 250 mL of dichloromethane and stirred. The solution was added sequentially with 0.9 g (15 mol %) of 4-dimethylaminopyridine, 7.2 g (1.2 equiv.) of pivaloyl chloride, and 10.5 mL (1.5 equiv.) of triethylamine and stirred at room temperature for 3 hours. When the reaction was completed, 600 mL of cold purified water was added and stirred for 30 minutes. The stirring was stopped, layers were separated, an organic layer was collected, and an aqueous layer was extracted twice with 500 mL of dichloromethane.

The organic layer was washed with water, brine, and a saturated sodium hydrogen carbonate solution, respectively, dried using sodium sulfate, and the solvent was concentrated under reduced pressure to obtain a crude compound. The crude compound was recrystallized by adding hexane to obtain 18.1 g (75%) of the labeled compound (E)-(3R,3aS,6R,7R,8aS)-3,6,8,8-tetramethyloctahydro-1H-3a,7-methanoazulen-6-yl-3-(3-methoxy-4-(pivaloyloxy)phenyl)acrylate.

¹H-NMR (400 MHz, CDCl3) δ0.89 (9H), 1.01 (1H), 1.15 (1H), 1.31 (10H), 1.41 (5H), 1.56 (2H), 1.65 (3H), 1.90 (3H), 3.87 (3H), 6,3 (1H), 7.3 (2H), 7.5 (1H), 7.6 (2H).

### 1.25. Preparation of (E)-(3R,3aS,6R,7R,8aS)-3,6,8,8-tetramethyloctahydro-1H-3a,7-methanoazulen-6-yl-3-(3-hydroxy-4-piyaloyloxy)phenyl)acrylate [Compound 1-25]

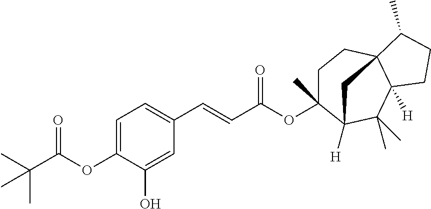 [SMILES: C[C@@H]1CC[C@H]2C(C)(C)[C@H]3C[C@@]12CC[C@@]3(C)OC(=O)/C=C/c1ccc(OC(=O)C(C)(C)C)c(O)c1]

24.1 g (0.05 mol, 1 equiv.) of (E)-(3R,3a8,6R,7R,8aS)-3,6,8,8-tetramethyloctahydro-1H-3a,7-methanoazulen-6-yl-3-(3-methoxy-4-(pivaloyloxy)phenyl)acrylate prepared in Example 24 was added in 250 mL of dichloromethane and stirred. The solution was added with 12.5 g (1 equiv.) of boron tribromide and stirred at room temperature for 1 hour. When the reaction was completed, 500 mL of cold purified water was added and stirred for 30 minutes. The stirring was stopped, layers were separated, and an aqueous layer was extracted twice with 500 mL of dichloromethane. An organic layer was collected, washed twice with 500 ml of purified water, and then dried using sodium sulfate, and the solvent was concentrated under reduced pressure to obtain a crude compound. The crude compound was purified by column chromatography (ethyl acetate:hexane=1:3) to obtain 17.6 g (75%) of the labeled compound (E)-(3R,3a8,6R,7R,8aS)-3,6,8,8-tetramethyloctahydro-1H-3a,7-methanoazulen-6-yl-3-(3-hydroxy-4-(pivaloyloxy)phenyl)acrylate.

¹H-NMR (400 MHz, CDCl3) δ0.89 (9H), 1.01 (1H), 1.15 (1H), 1.31 (10H), 1.41 (5H), 1.56 (2H), 1.65 (3H), 1.90 (3H), 6,3 (1H), 7.3 (2H), 7.5 (1H), 7.6 (2H), 9.48 (1H).

### 1.26. Preparation of 2-methoxy-5-((E)-3-oxo-3-(((3R,3aS,6R,7R,8aS)-3,6,8,8-tetramethyloctahydro-1H-3a,7-methanoazulen-6-yl)oxy)propen-1-yl)phenyl L-leucinate [Compound 1-26]

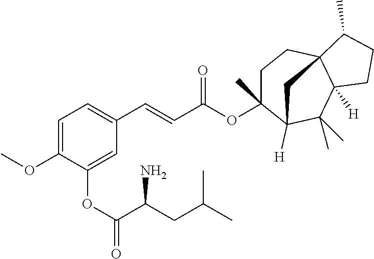 [SMILES: COc1ccc(/C=C/C(=O)O[C@]2(C)CC[C@@]34C[C@@H]2C(C)(C)[C@@H]3CC[C@H]4C)cc1OC(=O)[C@@H](N)CC(C)C]

2.5 g (0.01 mol, 1 equiv.) of (E)-3-(3-hydroxy-4-methoxyphenyl)acrylate and 3.4 g (1.2 equiv.) of (3R,3aS,6R,7R,8aS)-3,6,8,8-tetramethyloctahydro-1H-3a,7-methanoazulen-6-ol were added to 60 ml of tetrahydrofuran and stirred and dissolved. The solution was added with 1 mL of sulfuric acid, stirred for 30 minutes, and then added with 7.0 g of magnesium sulfate and the reaction solution was heated and refluxed for 8 hours. When the reaction was completed, the reaction solution was concentrated under reduced pressure to remove tetrahydrofuran, and added with 250 mL of ethyl acetate and 250 mL of a saturated sodium hydrogen carbonate solution and stirred for 30 minutes. The stirring was stopped, layers were separated, and an aqueous layer was extracted twice with 250 mL of dichloromethane. An organic layer was collected, washed using water and brine, and then dried using sodium sulfate, and the solvent was concentrated under reduced pressure to obtain (E)-(3R,3aS,6R,7R,8aS)-3,6,8, 8-tetramethylo ctahydro-1H-3a,7-methano azulen-6-yl-3-(3 -Hydroxy-4-methoxyphenyl)acrylate in a concentrate state. In a separate reactor, the concentrate was added with 1.7 g (0.01 mol, 1 equiv.) of L-leucine and 60 mL of dichloromethane, and added with 2.5 g (1.01 equiv.) of p-toluenesulfonyl chloride, and then refluxed at 40° C. for 3 hours. After the reaction was completed, the reaction solution was cooled to room temperature and then added to the concentrate of (E)-(3R,3aS,6R,7R,8aS)-3,6,8, 8-tetramethylo ctahydro-1H-3a,7-methano azulen-6-yl-3-(3 -Hydroxy-4-methoxyphenyl)acrylate and refluxed at 40° C. for 5 hours. After the reaction was completed, the reaction solution was added with 100 mL of water, stirred for 30 minutes, and left, and then the organic layer was washed using water and brine, respectively, dried using sodium sulfate, and the solvent was concentrated under reduced pressure to obtain a crude compound. The crude compound was purified by column chromatography (ethyl acetate:hexane=1:3) to obtain 3.8 g (57%) of the labeled compound (2-methoxy-5-((E)-3-oxo-3-(((3R,3aS,6R,7R,8aS)-3,6,8,8-tetramethyloctahydro-1H-3a,7-methanoazulen-6-yl)oxy)propen-1-yl)phenyl L-leucinate.

¹H-NMR(400 MHz, CDCl3) δ0.80-1.00 (15H), 1.20-1.50 (10H), 1.80-1.90 (8H), 3.36 (1H), 3.83 (3H), 6.31 (1H), 6.90-7.00 (2H), 7.33 (1H), 7.48 (1H), 8.76 (2H).

### 1.27. Preparation of (3R,3aS,6R,7R,8aS)-3,6,8,8-tetramethyloctahydro-1H-3a,7-methanoazulen-6-yl (E)-3-(4-acetoxy-3-methoxyphenyl)acrylate [Compound 1-27]

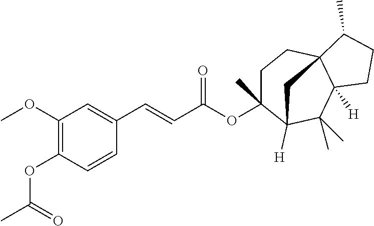 [SMILES: COc1cc(/C=C/C(=O)O[C@]2(C)CC[C@@]34C[C@@H]2C(C)(C)[C@@H]3CC[C@H]4C)ccc1OC(C)=O]

2.0 g (0.01 mol, 1 equiv.) of (E)-3-(3-hydroxy-4-methoxyphenyl)acrylate and 2.7 g (1.2 equiv.) of (3R,3aS,6R,7R,8aS)-3,6,8,8-tetramethyloctahydro-1H-3a,7-methanoazulen-6-ol were added to 60 ml of tetrahydrofuran and stirred and dissolved. The solution was added with 1 mL of sulfuric acid, stirred for 30 minutes, and then added with 4.8 g of magnesium sulfate and the reaction solution was heated and refluxed for 8 hours. When the reaction was completed, the reaction solution was cooled to room temperature, and 1.1 g (1.0 equiv.) of acetic anhydride was added to the reaction solution and stirred for 2 hours. When the reaction was completed, the reaction solution was concentrated under reduced pressure to remove tetrahydrofuran, and added with 200 mL of ethyl acetate and 200 mL of a saturated sodium hydrogen carbonate solution and stirred for 30 minutes. The stirring was stopped, layers were separated, and then the reaction solution was washed with water and brine, respectively, dried using sodium sulfate, and the solvent was concentrated under reduced pressure to obtain a crude compound. The crude compound was purified by column chromatography (ethyl acetate:hexane=1:1) to obtain 2.7 g (60%) of the labeled compound (3R,3aS,6R,7R,8aS)-3,6,8,8-tetramethyloctahydro-1H-3a,7-methanoazulen-6-yl (E)-3-(4-acetoxy-3-methoxyphenyl)acrylate.

¹H-NMR(400 MHz, CDCl3) δ0.80-0.90 (9H), 1.01 (1H), 1.10-1.60(10H), 1.65-1.90 (4H), 2.28 (3H), 3.87 (3H), 6.31 (1H), 7.10 (1H), 7.20-7.30 (2H), 7.48 (1H).

### 1.28. Preparation of (3R,3aS,6R,7R,8aS)-3,6,8,8-tetramethyloctahydro-1H-3a,7-methanoazulen-6-yl (E)-3-(4-isopropoxy-3-methoxyphenyl)acrylate [Compound 1-28]

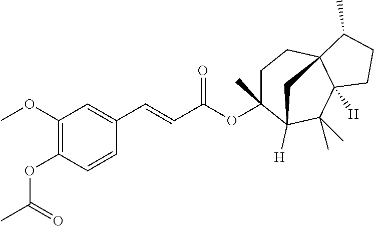 [SMILES: COc1cc(/C=C/C(=O)O[C@]2(C)CC[C@@]34C[C@@H]2C(C)(C)[C@@H]3CC[C@H]4C)ccc1OC(C)=O]

2.0 g (0.01 mol, 1 equiv.) of (E)-3-(3-hydroxy-4-methoxyphenyl)acrylic acid and 2.7 g (1.2 equiv.) of (3R,3aS,6R,7R,8aS)-3,6,8,8-tetramethyloctahydro-1H-3a,7-methanoazulen-6-ol were added to 60 ml of tetrahydrofuran and stirred and dissolved. The solution was added with 1 mL of sulfuric acid, stirred for 30 minutes, and then added with 4.8 g of magnesium sulfate and the reaction solution was heated and refluxed for 8 hours. When the reaction was completed, the reaction solution was cooled to room temperature, and 3.8 mL (4.0 equiv.) of 2-propane chloride was added to the reaction solution and stirred for 5 hours. When the reaction was completed, the reaction solution was concentrated under reduced pressure to remove tetrahydrofuran, and added with 200 mL of ethyl acetate and 200 mL of a saturated sodium hydrogen carbonate solution and stirred for 30 minutes. The stirring was stopped, layers were separated, and then the reaction solution was washed with water and brine, respectively, dried using sodium sulfate, and the solvent was concentrated under reduced pressure to obtain a crude compound. The crude compound was purified by column chromatography (ethyl acetate:hexane=1:1) to obtain 2.9 g (64%) of the labeled compound (3R,3aS,6R,7R,8aS)-3,6,8,8-tetramethyloctahydro-1H-3a,7-methanoazulen-6-yl (E)-3-(4-isopropoxy-3-methoxyphenypacrylate.

¹H-NMR(400 MHz, CDCl3) δ0.80-0.90 (9H), 1.01 (1H), 1.10-1.60 (11H), 1.65-1.90 (6H), 3.87 (3H), 4.67 (1H), 6.31 (1H), 6.97 (1H), 7.05 (1H), 7.23 (1H), 7.48 (1H).

### 1.29. Preparation of (3R,3aS,6R,7R,8aS)-3,6,8,8-tetramethyloctahydro-1H-3a,7-methanoazulen-6-yl (E)-3-(3-methoxy-4-((2-(4-((2-oxocyclopentyl)phenyl)propanoyl)oxy)phenyl)acrylate [Compound 1-29]

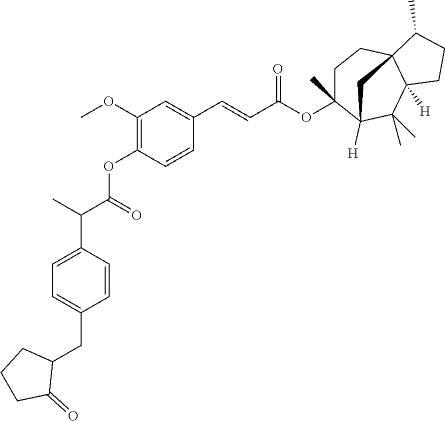 [SMILES: COc1cc(/C=C/C(=O)O[C@]2(C)CC[C@@]34C[C@@H]2C(C)(C)[C@@H]3CC[C@H]4C)ccc1OC(=O)C(C)c1ccc(CC2CCCC2=O)cc1]

4.0 g (0.02 mol, 1.0 equiv.) of 2-(4-((2-oxocyclopentyl)-methyl)phenyl)propanoic acid and 40 mL of dichloromethane were added to a reactor, and 2.0 g (1.02 equiv.) of thionyl chloride was added dropwise to the reactor. After stirring at room temperature for 2 hours, the reaction solution was concentrated under reduced pressure to remove dichloromethane, and then added with 60 ml of tetrahydrofuran, and added with 6.4 g (0.02 mol, 1.0 equiv.) of (E)-(3R,3aS,6R,7R,8aS)-3,6,8,8-tetramethyloctahydro-1H-3a,7-methanoazulen-6-yl-3-(4-hydroxy-3-methoxyphenyl)acrylate prepared in Example 8 and stirred at 40° C. for 3 hours. When the reaction was completed, the reaction solution was concentrated under reduced pressure to remove tetrahydrofuran, and added with 300 mL of ethyl acetate and 300 mL of water and stirred for 30 minutes. The stirring was stopped, an organic layer was washed with water and brine, respectively, dried using sodium sulfate, and the solvent was concentrated under reduced pressure to obtain a crude compound. The crude compound was purified by column chromatography (ethyl acetate:hexane=1:1) to obtain 5.2 g (51%) of the labeled compound (3R,3a8,6R,7R,8aS)-3,6,8,8-tetramethyloctahydro-1H-3a,7-methanoazulen-6-yl (E)-3-(3-methoxy-4-((2-(4-((2-oxocyclopentyl)phenyl)propanoyl)oxy)phenyl)acrylate.

¹H-NMR(400 MHz, CDCl3) δ0.80-0.90 (9H), 1.01 (1H), 1.10-1.39 (8H), 1.50-1.90 (6H), 1.92-2.10 (4H), 2.20-2.50 (3H), 2.85-3.10 (2H), 3.71 (1H), 3.87 (3H), 6.31 (1H), 7.05-7.30 (7H), 7.48 (1H).

### EXPERIMENTAL EXAMPLE 1. EXPERIMENT ON SARCOPENIA INHIBITORY EFFECT OF SESQUITERPENE DERIVATIVE

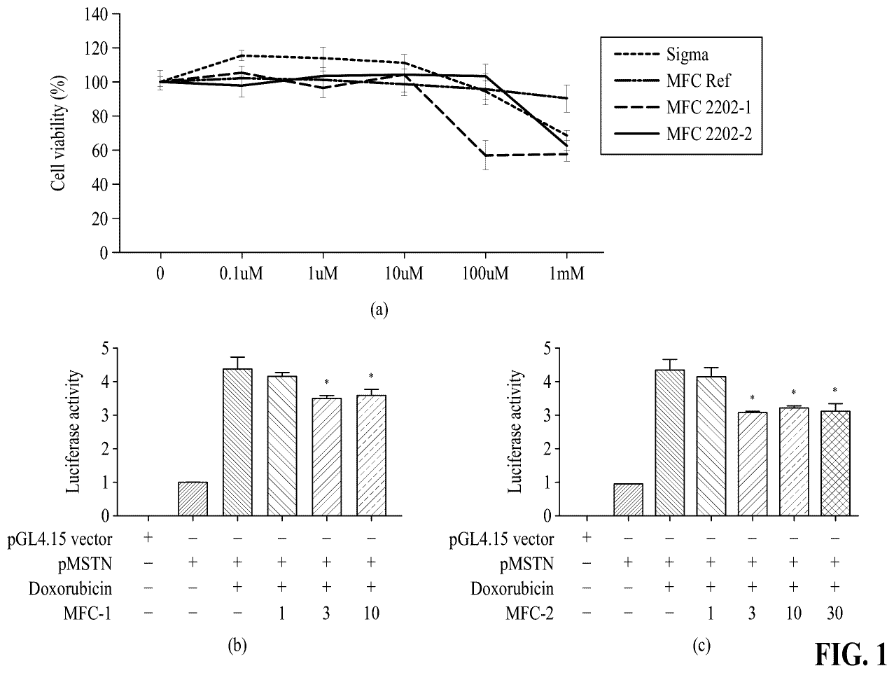

Cedrol (MFC Reference), Compound 1-1 (MFC-1), and Compound 1-2 (MFC-2) were each treated at concentrations of 0, 0.1 μM, 1 μM, 10 μM, 100 μM, and 1 mM for 24 hours, and then MTT assay was performed, and the cell viability results at each concentration were illustrated in FIG. 1A.

After 12 hours of transfection with 1 μg of a pMSTN_luc vector (myostatin; new plasmid vector generated by inserting a promoter region of a MSTN gene into a plasmid vector (pGL4.15 vector) capable of luciferase experiment), Compound 1-1 (MFC-1) was pretreated (1, 3, and 10 μM) for 3 hours, respectively. Thereafter, luciferase activity was measured by harvesting after treatment with 0.5 μg/ml of an anticancer agent Doxorubicin for 6 hours. The results were illustrated in FIG. 1B.

As may be seen in FIG. 1B, when the anticancer agent Doxorubicin was treated, the activity of the promoter of MSTN, a gene causing sarcopenia was rapidly increased, but since the MSTN promoter activity in a group treated with Doxorubicin after pretreatment with Compound 1-1 (MFC-1) showed a lower expression level than a group treated with Doxorubicin alone, it may be seen that Compound 1-1 (MFC-1) may reduce the MSTN promoter activity caused by the anticancer agent Doxorubicin. (Meanwhile, the luciferase assay used in FIG. 1B was an assay of transfecting a luciferase vector into a cell, treating a drug, and then treating luciferin to measure the amount of light emitted by the activation of the promoter of the gene by the drug. This means that it is possible to determine whether the promoter was activated and that the gene may be regulated at the transcriptional level by the drug touching the promoter region.)

After 12 hours of transfection with 1 μg of the pMSTN_luc vector, Compound 1-2 (MFC-2) was pretreated (1, 3, 10, and 30 μM) for 3 hours, respectively. Thereafter, the luciferase activity was measured by harvesting after treatment with Doxorubicin 0.5 μg/ml for 6 hours. The results were illustrated in FIG. 1C.

As may be seen in FIG. 1C, it may be seen that since in a group pretreated with Compound 1-2 (MFC-2) and treated with Doxorubicin, the MSTN promoter activity showed a lower expression level than the group treated with Doxorubicin alone, it may be seen that Compound 1-2 (MFC-2) may reduce the MSTN promoter activity caused by the anticancer drug Doxorubicin, and Compound 1-2 (MFC-2) has a greater inhibitory effect than Compound 1-1 (MFC-1).

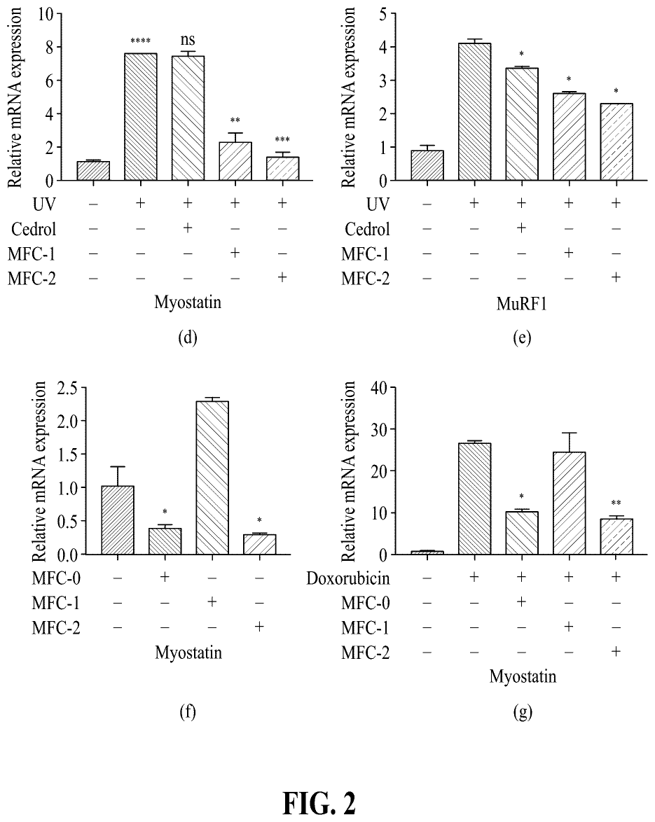

Cedrol, Compound 1-1 (MFC-1), and Compound 1-2 (MFC-2) were each pretreated (3 μM) for 3 hours, irradiated with 3 J/m² of UV, and then harvested, and myostatin was confirmed by performing qRT-PCR (FIG. 2D).

Cedrol, Compound 1-1 (MFC-1), and Compound 1-2 (MFC-2) were each pretreated (3 μM) for 3 hours, irradiated with 3 J/m² of UV, and then harvested, and MURF-1 was confirmed by performing qRT-PCR (FIG. 2E).

In cells of a basal condition, Cedrol (MFC-0), Compound 1-1 (MFC-1), and Compound 1-2 (MFC-2) were each pretreated (3 μM) for 12 hours, and harvested, and myostatin was confirmed by performing qRT-PCR (FIG. 2F).

Cedrol (MFC-0, Reference), Compound 1-1 (MFC-1), and Compound 1-2 (MFC-2) were each pretreated (3 μM) for 3 hours, treated with 0.5 μg/ml of Doxorubicin for 6 hours, and then harvested, and myostatin was confirmed by performing qRT-PCR (FIG. 2G).

### EXPERIMENTAL EXAMPLE 2. IDENTIFICATION OF MECHANISM OF COMPOUND 1-3 (MFC-2)

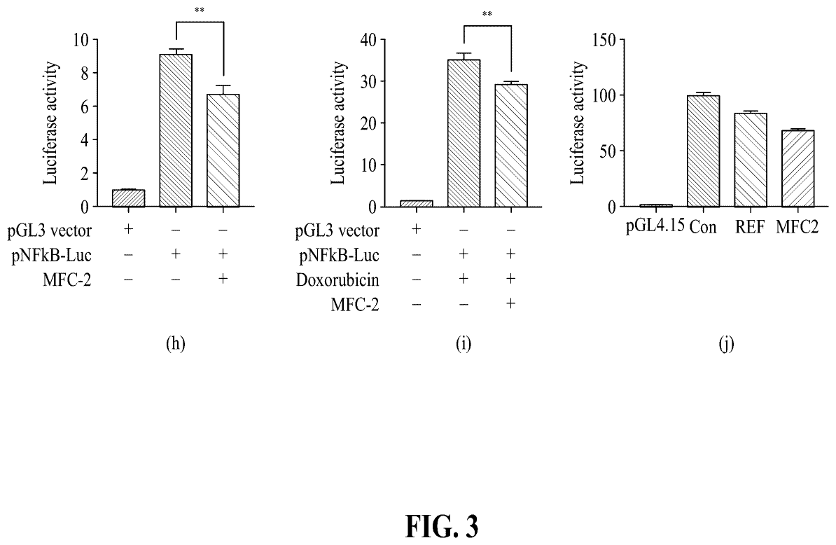

NF-κB was a transcription factor binding to a promoter region of a myostatin gene, a muscle mass regulation target protein, and to determine whether NF-κB was involved in the regulation of myostatin expression of Compound MFC-2, after 12 hours of transfection with 1 μg of the pNF-κB vector, Compound 1-2 (MFC-2) was treated (3 μM) for 12 hours. Thereafter, the luciferase activity was measured by harvesting (FIG. 3H). For reference, pGL3 and pGL4.15 shown in FIG. 3 were a type of luciferase plasmid vector, and the vector itself without the gene inserted was used as Control (control group). The vector in which the promoter region of the NF-κB gene was inserted into the luciferase vector was expressed as pNF-κB.

As illustrated in FIG. 3H, the promoter activity of pNF-κB was confirmed after pretreatment with Compound 1-2 (MFC-2), and as a result, it may be seen that MFC-2 reduces NF-κB promoter activity.

After 12 hours of transfection with 1 μg of the pNF-κB vector, Compound 1-2 (MFC-2) was pretreated (3 μM) for 3 hours. Thereafter, 0.5 μg/ml of Doxorubicin was treated for 6 hours, harvested, and luciferase activity was measured (FIG. 3I).

As may be seen from FIG. 3I, when the anticancer agent Doxorubicin was treated, the promoter activity of NF-κB, a transcription factor for MSTN, a gene causing sarcopenia, is increased, and in a group pretreated with Compound 1-2 (MFC-2) and treated with Doxorubicin, the NF-κB promoter activity showed a lower expression level than the group treated with Doxorubicin alone, and as a result, it may be seen that Compound 1-2 (MFC-2) may reduce the NF-κB promoter activity up-regulated by the anticancer agent Doxorubicin.

Cedrol (Reference) and Compound 1-2 (MFC-2) were each treated (3 μM), respectively, and harvested 12 hours later to measure luciferase activity (FIG. 3J).

As may be seen from the result in FIG. 3J, it may be seen that the group treated with Compound 1-2 (MFC-2) reduced pMSTN-luciferase activity more effectively than existing Cedrol (Reference).

### EXPERIMENTAL EXAMPLE 3. ANIMAL EXPERIMENT OF AGING MICE

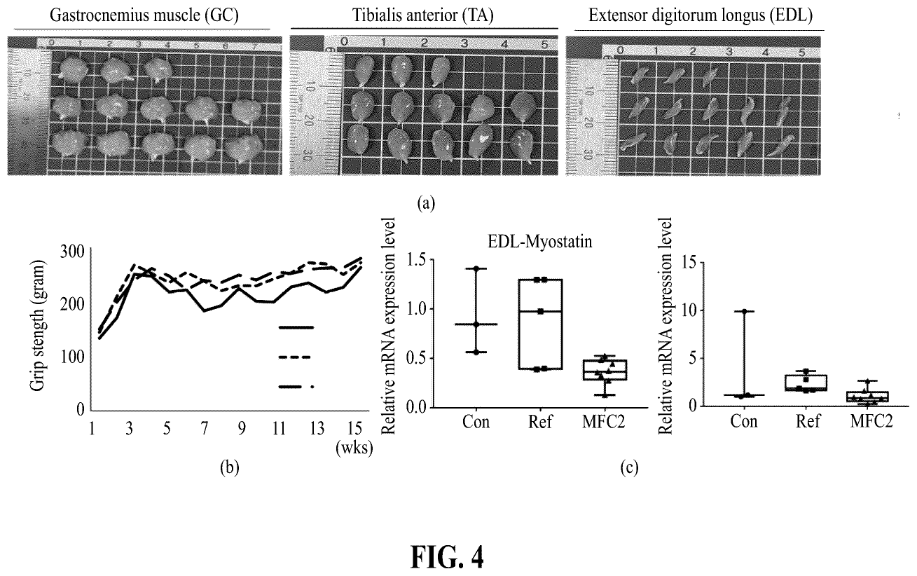

21-month-old aging mice were fed with regular feed (Control), feed containing Cedrol (Reference), and feed containing Compound 1-2 (MFC-2) for 3 months for each group, and thereafter, an animal experiment was conducted to compare the sizes of the Gastrocnemius muscle (GC), Tibialis anterior (TA), and Extensor digitorum longus (EDL) muscles separated from the hind limbs of aging mice. Through GC, TA, and EDL muscle images (FIG. 4A), it was confirmed that an increase in muscle size was confirmed in the MFC-2 group compared to the Control group.

In addition, the muscle strengths of the forelimbs and hindlimbs of 24 to 25-month-old mice were evaluated for the three groups using a grip strength measuring machine, and the evaluation was conducted once a week for 16 weeks. As a result of the experiment, it was confirmed that the strength of the MFC-2 group increased compared to the Control group (FIG. 4B).

As may be seen in FIG. 4B, as a result of the experiment using the grip strength machine, it may be seen that the muscle strengths of the Reference group (Cedrol) and the MFC-2 group gradually increased compared to the Control group, and in addition, it may be seen that the muscle strength of the MFC-2 group increased compared to the Reference group from week 15 later.

Meanwhile, qRT-PCR was performed using EDL muscle tissue, and as illustrated in FIG. 4C, it was confirmed that myostatin expression in the MFC-2 group was significantly decreased compared to the Control group.

As illustrated in FIG. 4C, RNA was extracted from EDL muscle tissue separated from the hind limbs of aging mice by crushing the EDL muscle tissue at a predetermined temperature, CDNA was synthesized, and the expression level of mRNA was confirmed using the REALTIME PCR technique. MuRF1 (muscle RING-finger protein 1) was a muscle-specific E3 ubiquitin ligase and a type of atrogene causing sarcopenia, and it was confirmed that the expression of Myostatin and MuRF1 was reduced in the EDL muscle tissue of the group fed with Compound MFC-2.

### EXPERIMENTAL EXAMPLE 4. ANIMAL BLOOD ANALYSIS EXPERIMENT OF AGING MICE

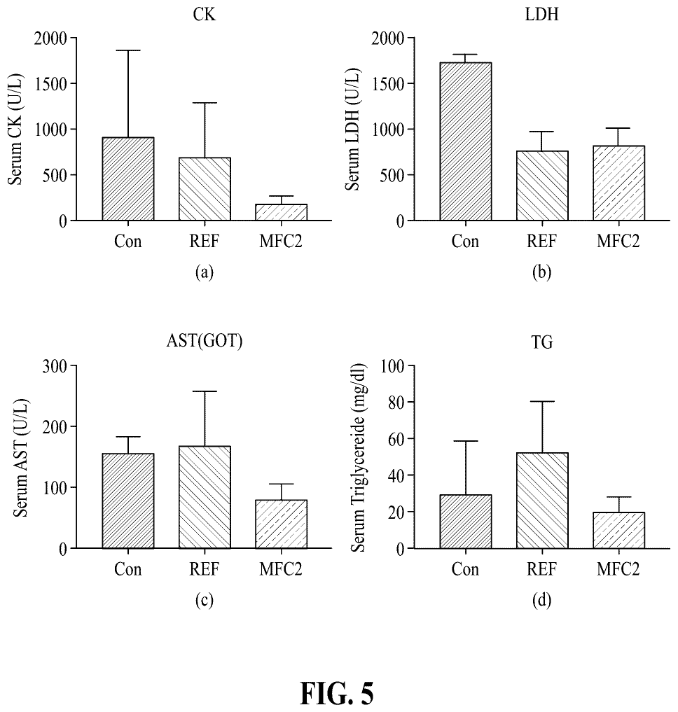

For the Control group, Reference group, and MFC-2 group, an animal blood analysis experiment was conducted on aging mice. As a result, it was confirmed that the serum CK level of creatine kinase (CK), which was an enzyme contained in skeletal muscle, myocardium, smooth muscle, etc. to be released into the blood when the muscle was damaged, decreased in the MFC-2 group compared to other groups (FIG. 5A).

In addition, the experiment was conducted in the same manner even on Lactate dehydrogenase (LDH), which was released during cell destruction and was an indicator of sarcopenia, and as a result, a decrease in LDH level was confirmed in the MFC-2 group compared to the Control group (FIG. 5B).

In addition, even in AST (Aspartate transaminase) and TG (Triglyceride), as indicators related to muscle function, a decrease in the levels thereof was confirmed in the MFC-2 group compared to other groups (FIGS. 5C and 5D).

The results of Examples suggest that the sesquiterpene derivative of the present disclosure may be effectively used to prevent or treat sarcopenia by inhibiting the expression of myostatin mRNA in muscle cells.

As described above, although the embodiments have been described by the restricted drawings, various modifications and variations may be applied on the basis of the embodiments by those skilled in the art. For example, even if the described techniques are performed in a different order from the described method, and/or components such as a system, a structure, a device, a circuit, and the like described above are coupled or combined in a different form from the described method, or replaced or substituted by other components or equivalents, an appropriate result may be achieved.

Therefore, other implementations, other embodiments, and equivalents to the appended claims fall within the scope of the claims to be described below.

### INDUSTRIAL APPLICABILITY

The present disclosure relates to a sesquiterpene derivative or a pharmaceutically acceptable salt thereof, a composition for preventing, ameliorating or treating sarcopenia, including the derivative or salt thereof as an active ingredient, and the like. The sesquiterpene derivative or pharmaceutically acceptable salt thereof of the present disclosure inhibits increases in the production and mRNA expression of a myostatin protein which directly affects muscle loss and reduced muscle strength, and thus may exhibit a more fundamental effect of preventing or treating sarcopenia.

1. A sesquiterpene derivative represented by Chemical Formula 1 below or a pharmaceutically acceptable salt thereof: 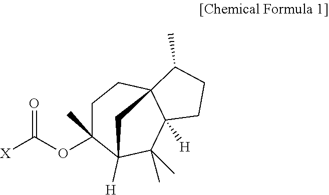
wherein,
X is 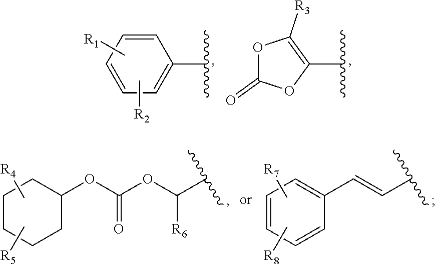
R₁ to R₈ are each independently hydrogen, halogen, straight or branched C1-C5 alkyl, straight or branched C2-C5 alkenyl, C2-C5 alkynyl, C3-C7 cycloalkyl, straight or branched C1-C5 alkoxy, —C(═O)Y₁, —OC(═O)Y₂, cyano, or hydroxyl,
wherein one or more hydrogens of the C1-C5 alkyl, C3-C7 cycloalkyl, or C1-C5 alkoxy are each independently unsubstituted or substituted with one or more substituents selected from the group consisting of halogen, hydroxy, cyano, nitro, NY₃Y₄, and 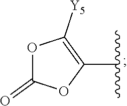
Y₁ to Y₅ are each independently hydrogen, straight or branched C1-C5 alkyl, or C3-C7 cycloalkyl (wherein one or more hydrogens of the C1-C5 alkyl or C3-C7 cycloalkyl are independently unsubstituted or substituted with one or more substituents selected from the group consisting of halogen, hydroxy, cyano, nitro, NZ₁Z₂, and 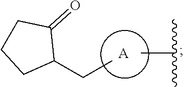
Z₁ and Z₂ are each independently hydrogen or straight or branched C1-C5 alkyl; and
A is a C3-C7 aryl group or a C3-C7 heteroaryl group,
wherein there are excluded:
1) (3R,3aS,6R,7R,8aS)-3,6,8,8-tetramethyloctahydro-1H-3a,7-metanoazulen-6-yl-4-hydroxy-3-methoxybenzoate;
2) (3R,3aS,6R,7R,8aS)-3,6,8,8-tetramethyloctahydro-1H-3a,7-metanoazulen-6-yl-4-acetoxy-3-methoxybenzoate;
3) (3R,3aS,6R,7R,8aS)-3,6,8,8-tetramethyloctahydro-1H-3a,7-methanoazulen-6-yl-3-methoxy-4-pivaloyloxybenzoate;
4) (3R,3aS,6R,7R,8aS)-3,6,8,8-tetramethyloctahydro-1H-3a,7-methanoazulen-6-yl-5-methyl-2-oxo-1,3-dioxole-4-carboxylate;
5) (3R,3aS,6R,7R,8aS)-3,6,8,8-tetramethyloctahydro-1H-3a,7-methanoazulen-6-yl-2-(((cyclohexyloxy)carbonyl)oxy)propanate;
6) (3R,3aS,6R,7R,8aS)-3,6,8,8-tetramethyloctahydro-1H-3a,7-metanoazulen-6-yl-4-(pivaloyloxy)benzoate;
7) (E)-(3R,3aS,6R,7R,8aS)-3,6,8,8-tetramethyloctahydro-1H-3a,7-methanoazulen-6-yl-3-(4-hydroxyphenyl)acrylate;
8) (E)-(3R,3aS,6R,7R,8aS)-3,6,8,8-tetramethyloctahydro-1H-3a,7-methanoazulen-6-yl-3-(4-hydroxy-3-methoxyphenyl)acrylate;
9) (E)-(3R,3aS,6R,7R,8aS)-3,6,8,8-tetramethyloctahydro-1H-3a,7-methanoazulen-6-yl-3-(3,4-dihydroxy phenyl)acrylate;
10) (E)-(3R,3aS,6R,7R,8aS)-3,6,8,8-tetramethyloctahydro-1H-3a,7-methanoazulen-6-yl cinnamate;
11) (E)-(3R,3aS,6R,7R,8aS)-3,6,8,8-tetramethyloctahydro-1H-3a,7-methanoazulen-6-yl-3-(4-(2-hydroxyethoxy)phenyl)acrylate; and
12) (E)-(3R,3aS,6R,7R,8aS)-3,6,8,8-tetramethyloctahydro-1H-3a,7-methanoazulen-6-yl-3-(4-(2-(dimethylamino)ethoxy)phenyl)acrylate.

2. The sesquiterpene derivative or the pharmaceutically acceptable salt thereof of claim 1, wherein in Chemical Formula 1,
R₁ to R₈ are each independently hydrogen, straight or branched C1-C5 alkyl, straight or branched C1-C5 alkoxy, —OC(═O)Y₂, cyano, or hydroxy,
wherein, one or more hydrogens of the C1-C5 alkoxy may be each independently unsubstituted or substituted with one or more substituents selected from the group consisting of hydroxy, NY₃Y₄, and 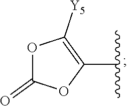
Y₂ to Y₅ are each independently straight or branched C1-C5 alkyl, or C3-C7 cycloalkyl, wherein, one or more hydrogens of the C1-C5 alkyl or C3-C7 cycloalkyl may be each independently unsubstituted or substituted with one or more substituents selected from the group consisting of NZ₁Z₂, and 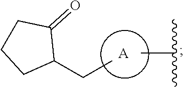
Z₁ and Z₂ are each independently hydrogen or straight or branched C1-C5 alkyl; and
A is a C3-C7 aryl group.

3. The sesquiterpene derivative or the pharmaceutically acceptable salt thereof of claim 1, wherein the sesquiterpene derivative represented by Chemical Formula 1 is selected from the group consisting of compounds represented by the following Chemical Formulas:

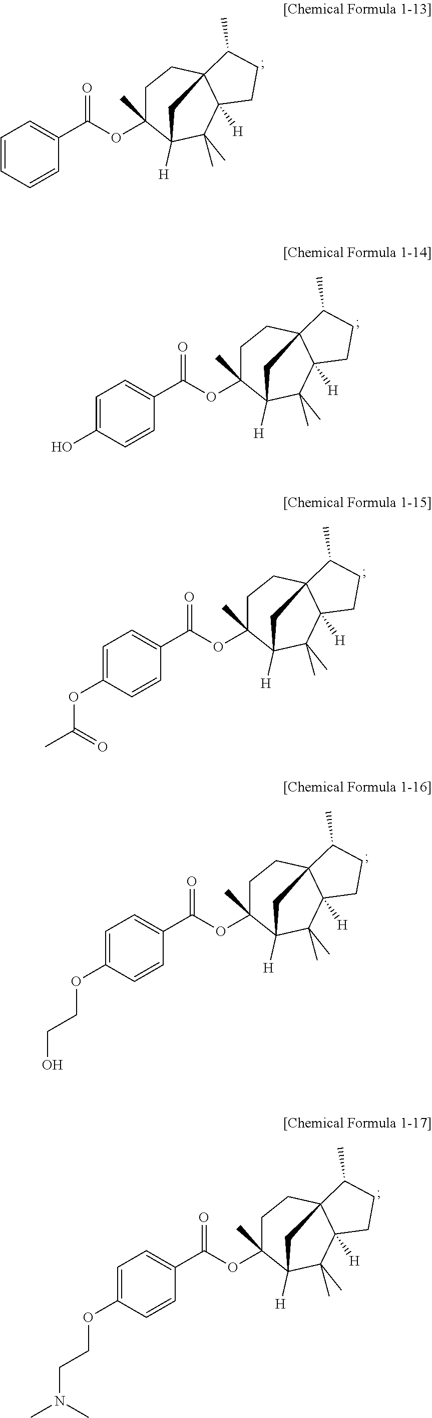 [SMILES: C[C@@H]1CC[C@H]2C(C)(C)[C@H]3C[C@@]12CC[C@@]3(C)OC(=O)c1ccccc1 ; C[C@@H]1CC[C@H]2C(C)(C)[C@H]3C[C@@]12CC[C@@]3(C)OC(=O)c1ccc(O)cc1 ; CC(=O)Oc1ccc(C(=O)O[C@]2(C)CC[C@@]34C[C@@H]2C(C)(C)[C@@H]3CC[C@H]4C)cc1 ; C[C@@H]1CC[C@H]2C(C)(C)[C@H]3C[C@@]12CC[C@@]3(C)OC(=O)c1ccc(OCCO)cc1 ; C[C@@H]1CC[C@H]2C(C)(C)[C@H]3C[C@@]12CC[C@@]3(C)OC(=O)c1ccc(OCCN(C)C)cc1]

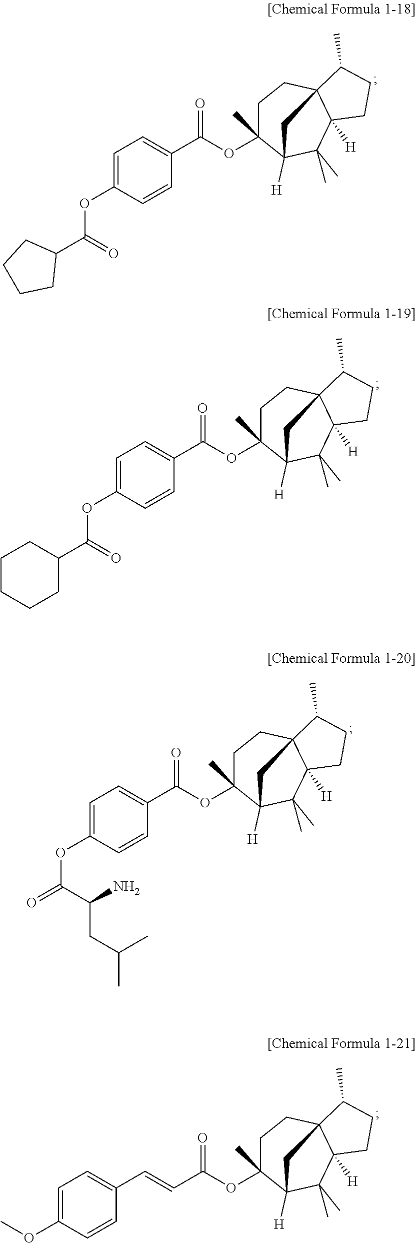 [SMILES: C[C@@H]1CC[C@H]2C(C)(C)[C@H]3C[C@@]12CC[C@@]3(C)OC(=O)c1ccc(OC(=O)C2CCCC2)cc1 ; C[C@@H]1CC[C@H]2C(C)(C)[C@H]3C[C@@]12CC[C@@]3(C)OC(=O)c1ccc(OC(=O)C2CCCCC2)cc1 ; CC(C)C[C@H](N)C(=O)Oc1ccc(C(=O)O[C@]2(C)CC[C@@]34C[C@@H]2C(C)(C)[C@@H]3CC[C@H]4C)cc1 ; COc1ccc(/C=C/C(=O)O[C@]2(C)CC[C@@]34C[C@@H]2C(C)(C)[C@@H]3CC[C@H]4C)cc1]

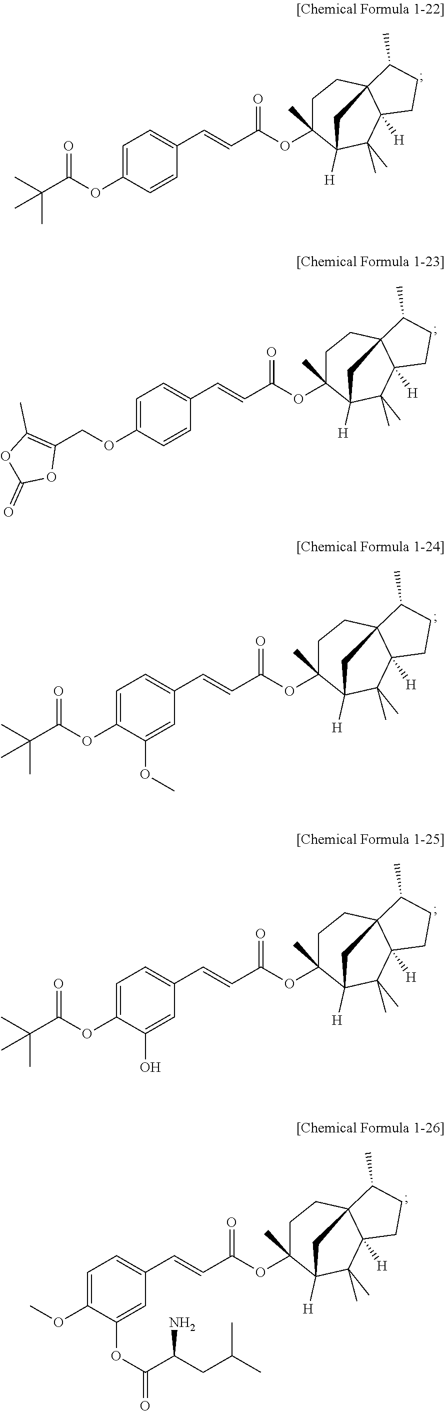 [SMILES: C[C@@H]1CC[C@H]2C(C)(C)[C@H]3C[C@@]12CC[C@@]3(C)OC(=O)/C=C/c1ccc(OC(=O)C(C)(C)C)cc1 ; Cc1oc(=O)oc1COc1ccc(/C=C/C(=O)O[C@]2(C)CC[C@@]34C[C@@H]2C(C)(C)[C@@H]3CC[C@H]4C)cc1 ; COc1cc(/C=C/C(=O)O[C@]2(C)CC[C@@]34C[C@@H]2C(C)(C)[C@@H]3CC[C@H]4C)ccc1OC(=O)C(C)(C)C ; C[C@@H]1CC[C@H]2C(C)(C)[C@H]3C[C@@]12CC[C@@]3(C)OC(=O)/C=C/c1ccc(OC(=O)C(C)(C)C)c(O)c1 ; COc1ccc(/C=C/C(=O)O[C@]2(C)CC[C@@]34C[C@@H]2C(C)(C)[C@@H]3CC[C@H]4C)cc1OC(=O)[C@@H](N)CC(C)C]

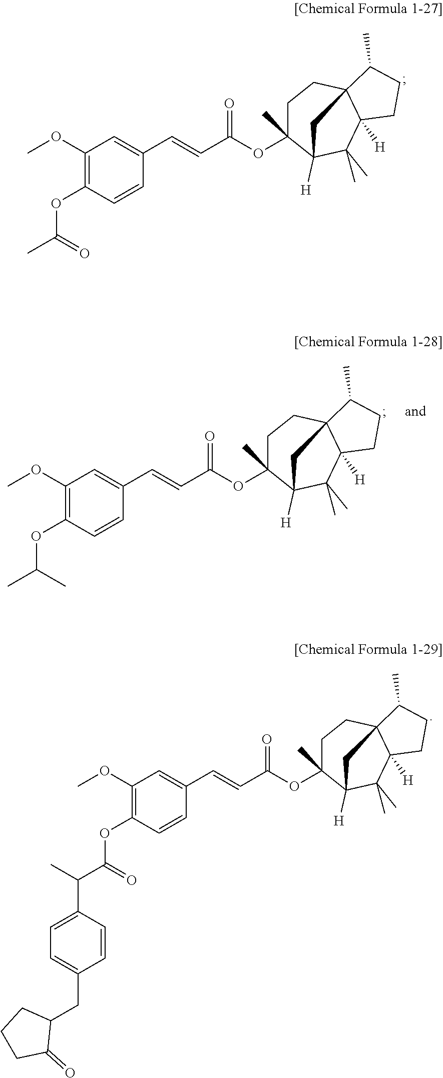 [SMILES: COc1cc(/C=C/C(=O)O[C@]2(C)CC[C@@]34C[C@@H]2C(C)(C)[C@@H]3CC[C@H]4C)ccc1OC(C)=O ; COc1cc(/C=C/C(=O)O[C@]2(C)CC[C@@]34C[C@@H]2C(C)(C)[C@@H]3CC[C@H]4C)ccc1OC(C)C ; COc1cc(/C=C/C(=O)O[C@]2(C)CC[C@@]34C[C@@H]2C(C)(C)[C@@H]3CC[C@H]4C)ccc1OC(=O)C(C)c1ccc(CC2CCCC2=O)cc1]

4. A pharmaceutical composition for preventing or treating sarcopenia comprising a sesquiterpene derivative represented by Chemical Formula 1 below or a pharmaceutically acceptable salt thereof as an active ingredient: 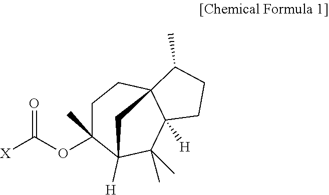
wherein,
X is 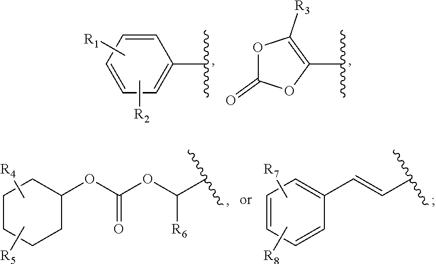
R₁ to R₈ are each independently hydrogen, halogen, straight or branched C1-C5 alkyl, straight or branched C2-C5 alkenyl, C2-C5 alkynyl, C3-C7 cycloalkyl, straight or branched C1-C5 alkoxy, —C(═O)Y₁, —OC(═O)Y₂, cyano, or hydroxyl, wherein one or more hydrogens of the C1-C5 alkyl, C3-C7 cycloalkyl, or C1-C5 alkoxy are each independently unsubstituted or substituted with one or more substituents selected from the group consisting of halogen, hydroxy, cyano, nitro, NY₃Y₄, and 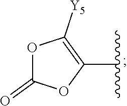
Y₁ to Y₅ are each independently hydrogen, straight or branched C1-C5 alkyl, or C3-C7 cycloalkyl, wherein one or more hydrogens of the C1-C5 alkyl or C3-C7 cycloalkyl are independently unsubstituted or substituted with one or more substituents selected from the group consisting of halogen, hydroxy, cyano, nitro, NZ₁Z₂, and 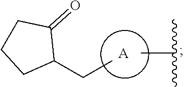
Z₁ and Z₂ are each independently hydrogen or straight or branched C1-C5 alkyl; and
A is a C3-C7 aryl group or a C3-C7 heteroaryl group.

5. The pharmaceutical composition for preventing or treating sarcopenia of claim 4, wherein the sesquiterpene derivative or the pharmaceutically acceptable salt thereof inhibits the expression of myostatin mRNA or protein.

6. The pharmaceutical composition for preventing or treating sarcopenia of claim 4, wherein the sesquiterpene derivative or the pharmaceutically acceptable salt thereof inhibits the expression of Muscle RING-finger protein-1 (MURF1) mRNA or protein or inhibits the expression of forkhead box O3 (Foxo3) mRNA or protein.

7. The pharmaceutical composition for preventing or treating sarcopenia of claim 4, wherein the sesquiterpene derivative represented by Chemical Formula 1 is selected from the group consisting of compounds represented by the following Chemical Formulas:

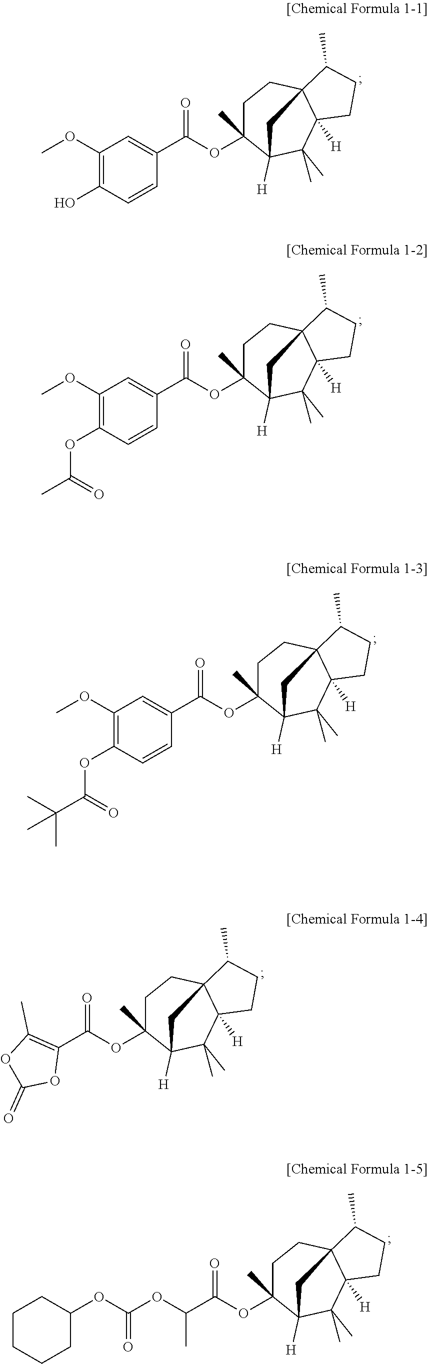 [SMILES: COc1cc(C(=O)O[C@]2(C)CC[C@@]34C[C@@H]2C(C)(C)[C@@H]3CC[C@H]4C)ccc1O ; COc1cc(C(=O)O[C@]2(C)CC[C@@]34C[C@@H]2C(C)(C)[C@@H]3CC[C@H]4C)ccc1OC(C)=O ; COc1cc(C(=O)O[C@]2(C)CC[C@@]34C[C@@H]2C(C)(C)[C@@H]3CC[C@H]4C)ccc1OC(=O)C(C)(C)C ; Cc1oc(=O)oc1C(=O)O[C@]1(C)CC[C@@]23C[C@@H]1C(C)(C)[C@@H]2CC[C@H]3C ; CC(OC(=O)OC1CCCCC1)C(=O)O[C@]1(C)CC[C@@]23C[C@@H]1C(C)(C)[C@@H]2CC[C@H]3C]

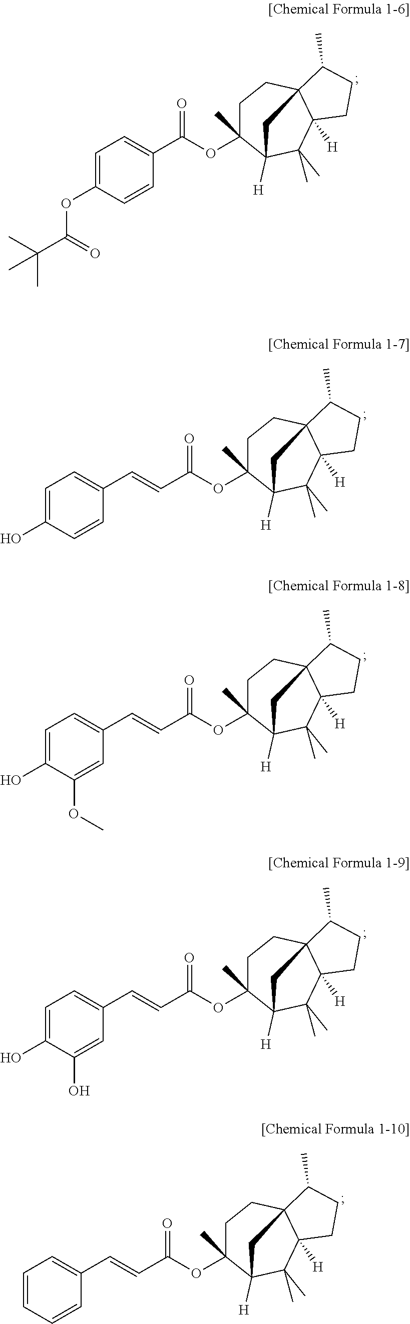 [SMILES: C[C@@H]1CC[C@H]2C(C)(C)[C@H]3C[C@@]12CC[C@@]3(C)OC(=O)c1ccc(OC(=O)C(C)(C)C)cc1 ; C[C@@H]1CC[C@H]2C(C)(C)[C@H]3C[C@@]12CC[C@@]3(C)OC(=O)/C=C/c1ccc(O)cc1 ; COc1cc(/C=C/C(=O)O[C@]2(C)CC[C@@]34C[C@@H]2C(C)(C)[C@@H]3CC[C@H]4C)ccc1O ; C[C@@H]1CC[C@H]2C(C)(C)[C@H]3C[C@@]12CC[C@@]3(C)OC(=O)/C=C/c1ccc(O)c(O)c1 ; C[C@@H]1CC[C@H]2C(C)(C)[C@H]3C[C@@]12CC[C@@]3(C)OC(=O)/C=C/c1ccccc1]

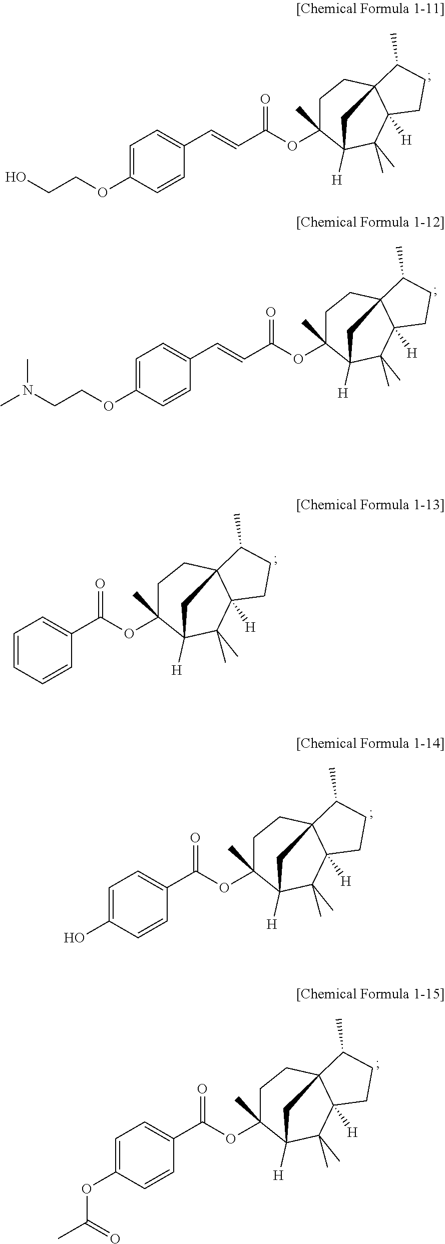 [SMILES: C[C@@H]1CC[C@H]2C(C)(C)[C@H]3C[C@@]12CC[C@@]3(C)OC(=O)/C=C/c1ccc(OCCO)cc1 ; C[C@@H]1CC[C@H]2C(C)(C)[C@H]3C[C@@]12CC[C@@]3(C)OC(=O)/C=C/c1ccc(OCCN(C)C)cc1 ; C[C@@H]1CC[C@H]2C(C)(C)[C@H]3C[C@@]12CC[C@@]3(C)OC(=O)c1ccccc1 ; C[C@@H]1CC[C@H]2C(C)(C)[C@H]3C[C@@]12CC[C@@]3(C)OC(=O)c1ccc(O)cc1 ; CC(=O)Oc1ccc(C(=O)O[C@]2(C)CC[C@@]34C[C@@H]2C(C)(C)[C@@H]3CC[C@H]4C)cc1]

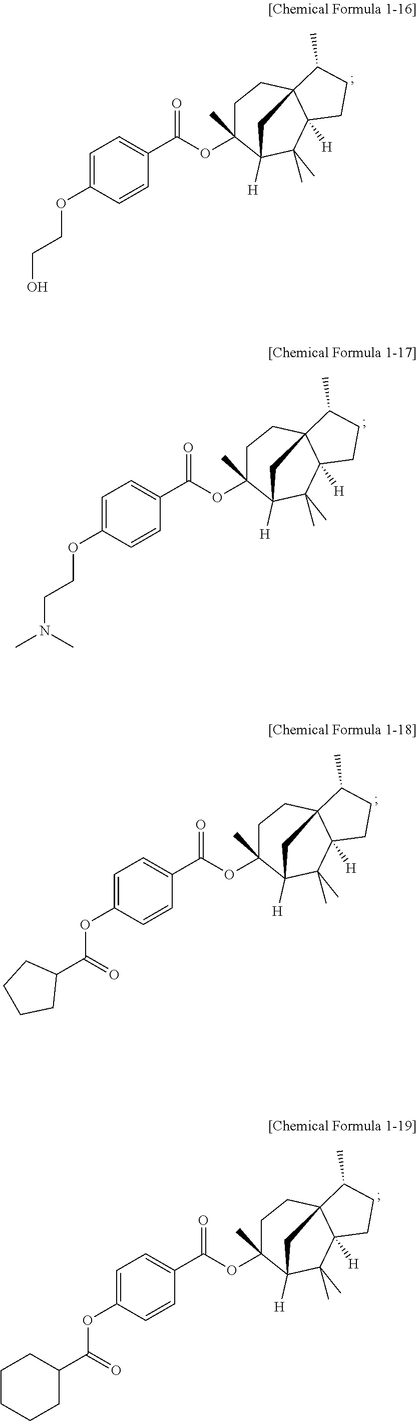 [SMILES: C[C@@H]1CC[C@H]2C(C)(C)[C@H]3C[C@@]12CC[C@@]3(C)OC(=O)c1ccc(OCCO)cc1 ; C[C@@H]1CC[C@H]2C(C)(C)[C@H]3C[C@@]12CC[C@@]3(C)OC(=O)c1ccc(OCCN(C)C)cc1 ; C[C@@H]1CC[C@H]2C(C)(C)[C@H]3C[C@@]12CC[C@@]3(C)OC(=O)c1ccc(OC(=O)C2CCCC2)cc1 ; C[C@@H]1CC[C@H]2C(C)(C)[C@H]3C[C@@]12CC[C@@]3(C)OC(=O)c1ccc(OC(=O)C2CCCCC2)cc1]

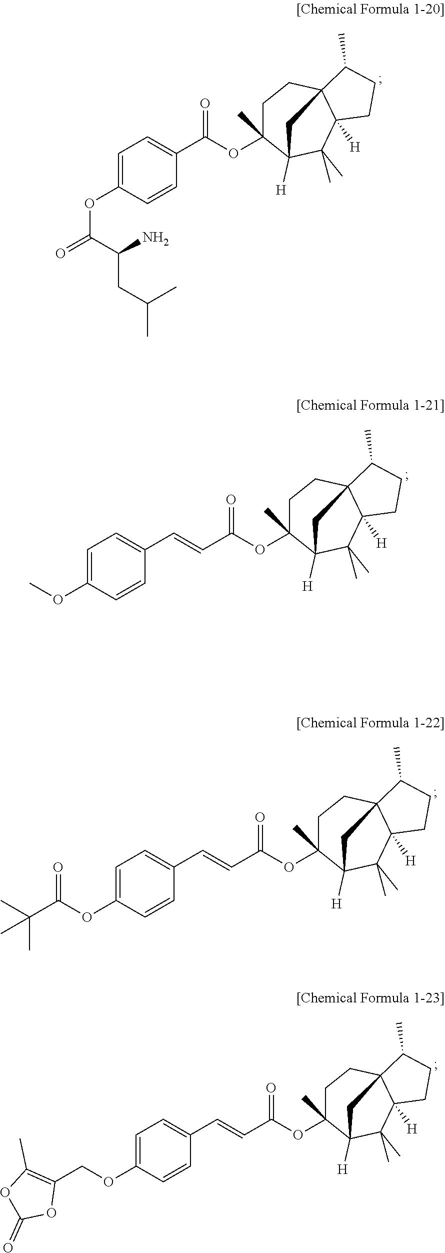 [SMILES: CC(C)C[C@H](N)C(=O)Oc1ccc(C(=O)O[C@]2(C)CC[C@@]34C[C@@H]2C(C)(C)[C@@H]3CC[C@H]4C)cc1 ; COc1ccc(/C=C/C(=O)O[C@]2(C)CC[C@@]34C[C@@H]2C(C)(C)[C@@H]3CC[C@H]4C)cc1 ; C[C@@H]1CC[C@H]2C(C)(C)[C@H]3C[C@@]12CC[C@@]3(C)OC(=O)/C=C/c1ccc(OC(=O)C(C)(C)C)cc1 ; Cc1oc(=O)oc1COc1ccc(/C=C/C(=O)O[C@]2(C)CC[C@@]34C[C@@H]2C(C)(C)[C@@H]3CC[C@H]4C)cc1]

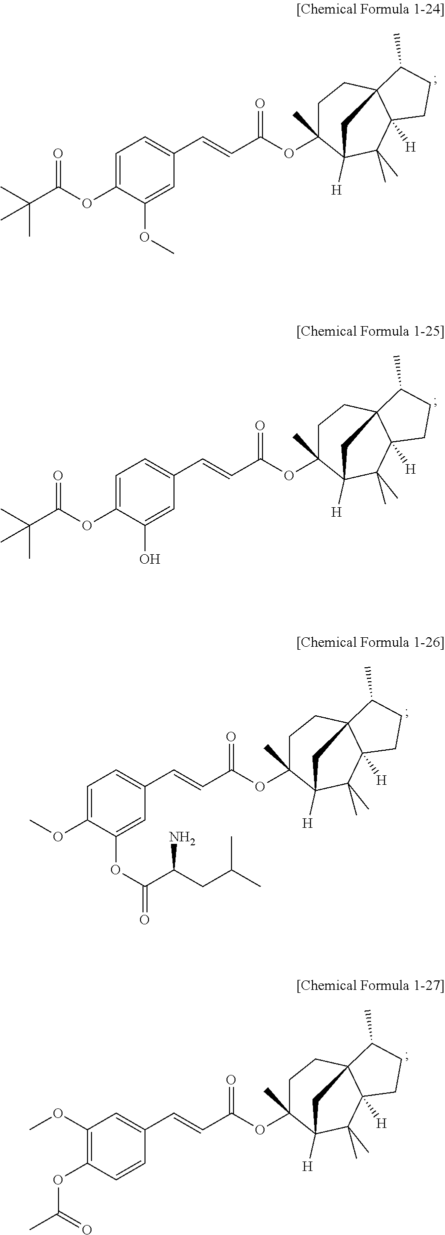 [SMILES: COc1cc(/C=C/C(=O)O[C@]2(C)CC[C@@]34C[C@@H]2C(C)(C)[C@@H]3CC[C@H]4C)ccc1OC(=O)C(C)(C)C ; C[C@@H]1CC[C@H]2C(C)(C)[C@H]3C[C@@]12CC[C@@]3(C)OC(=O)/C=C/c1ccc(OC(=O)C(C)(C)C)c(O)c1 ; COc1ccc(/C=C/C(=O)O[C@]2(C)CC[C@@]34C[C@@H]2C(C)(C)[C@@H]3CC[C@H]4C)cc1OC(=O)[C@@H](N)CC(C)C ; COc1cc(/C=C/C(=O)O[C@]2(C)CC[C@@]34C[C@@H]2C(C)(C)[C@@H]3CC[C@H]4C)ccc1OC(C)=O]

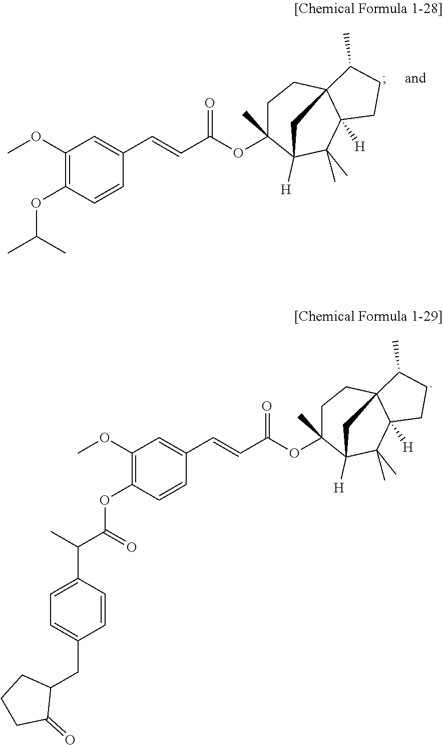 [SMILES: COc1cc(/C=C/C(=O)O[C@]2(C)CC[C@@]34C[C@@H]2C(C)(C)[C@@H]3CC[C@H]4C)ccc1OC(C)C ; COc1cc(/C=C/C(=O)O[C@]2(C)CC[C@@]34C[C@@H]2C(C)(C)[C@@H]3CC[C@H]4C)ccc1OC(=O)C(C)c1ccc(CC2CCCC2=O)cc1]

8. The pharmaceutical composition for preventing or treating sarcopenia of claim 4, further comprising:
the sesquiterpene derivative or a pharmaceutically acceptable salt thereof; and
one or more additional ingredients selected from the group consisting of pharmaceutically acceptable carriers, excipients, diluents, stabilizers and preservatives.

9. The pharmaceutical composition for preventing or treating sarcopenia of claim 8, wherein the pharmaceutical composition has formulations of powders, granules, tablets, capsules or injections.

10. A method for preventing or treating sarcopenia comprising administering a sesquiterpene derivative represented by Chemical Formula 1 below or a pharmaceutically acceptable salt thereof to a subject: 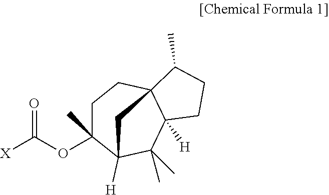
wherein,
X is 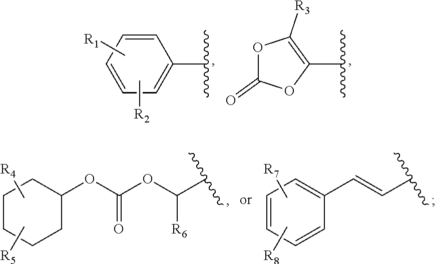
R₁ to R₈ are each independently hydrogen, halogen, straight or branched C1-C5 alkyl, straight or branched C2-C5 alkenyl, C2-C5 alkynyl, C3-C7 cycloalkyl, straight or branched C1-C5 alkoxy, —C(═O)Y₁, —OC(═O)Y₂, cyano, or hydroxyl, wherein one or more hydrogens of the C1-C5 alkyl, C3-C7 cycloalkyl, or C1-C5 alkoxy are each independently unsubstituted or substituted with one or more substituents selected from the group consisting of halogen, hydroxy, cyano, nitro, NY₃Y₄, and 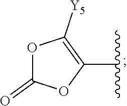
Y₁ to Y₅ are each independently hydrogen, straight or branched C1-C5 alkyl, or C3-C7 cycloalkyl, wherein one or more hydrogens of the C1-C5 alkyl or C3-C7 cycloalkyl are independently unsubstituted or substituted with one or more substituents selected from the group consisting of halogen, hydroxy, cyano, nitro, NZ₁Z₂, and 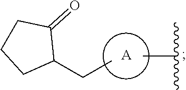
Z₁ and Z₂ are each independently hydrogen or straight or branched C1-C5 alkyl; and
A is a C3-C7 aryl group or a C3-C7 heteroaryl group.

11. A cosmetic composition for preventing or ameliorating sarcopenia comprising a sesquiterpene derivative represented by Chemical Formula 1 below or a cosmetically acceptable salt thereof as an active ingredient: 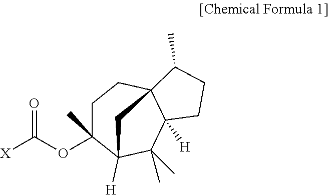
wherein,
X is 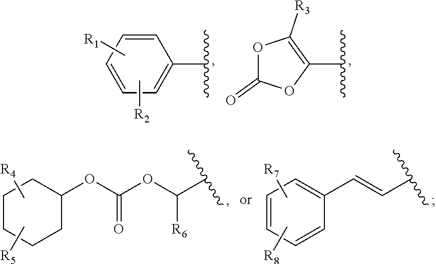
R₁ to R₈ are each independently hydrogen, halogen, straight or branched C1-C5 alkyl, straight or branched C2-C5 alkenyl, C2-C5 alkynyl, C3-C7 cycloalkyl, straight or branched C1-C5 alkoxy, —C(═O)Y₁, —OC(═O)Y₂, cyano, or hydroxyl, wherein one or more hydrogens of the C1-C5 alkyl, C3-C7 cycloalkyl, or C1-C5 alkoxy are each independently unsubstituted or substituted with one or more substituents selected from the group consisting of halogen, hydroxy, cyano, nitro, NY₃Y₄, and 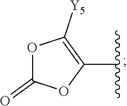
Y₁ to Y₅ are each independently hydrogen, straight or branched C1-C5 alkyl, or C3-C7 cycloalkyl, wherein one or more hydrogens of the C1-C5 alkyl or C3-C7 cycloalkyl are independently unsubstituted or substituted with one or more substituents selected from the group consisting of halogen, hydroxy, cyano, nitro, NZ₁Z₂, and 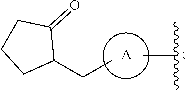
Z₁ and Z₂ are each independently hydrogen or straight or branched C1-C5 alkyl; and
A is a C3-C7 aryl group or a C3-C7 heteroaryl group.

12. A food composition for preventing or ameliorating sarcopenia comprising a sesquiterpene derivative represented by Chemical Formula 1 below or a food acceptable salt thereof as an active ingredient: 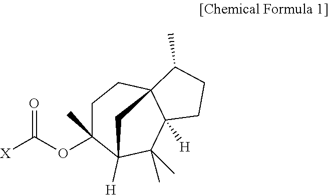
wherein,
X is 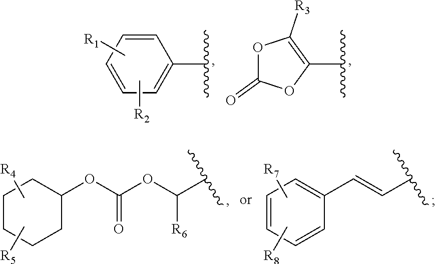
R₁ to R₈ are each independently hydrogen, halogen, straight or branched C1-C5 alkyl, straight or branched C2-C5 alkenyl, C2-C5 alkynyl, C3-C7 cycloalkyl, straight or branched C1-C5 alkoxy, —C(═O)Y₁, —OC(═O)Y₂, cyano, or hydroxyl, wherein one or more hydrogens of the C1-C5 alkyl, C3-C7 cycloalkyl, or C1-C5 alkoxy are each independently unsubstituted or substituted with one or more substituents selected from the group consisting of halogen, hydroxy, cyano, nitro, NY₃Y₄, and 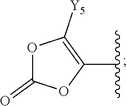
Y₁ to Y₅ are each independently hydrogen, straight or branched C1-C5 alkyl, or C3-C7 cycloalkyl, wherein one or more hydrogens of the C1-C5 alkyl or C3-C7 cycloalkyl are independently unsubstituted or substituted with one or more substituents selected from the group consisting of halogen, hydroxy, cyano, nitro, NZ₁Z₂, and 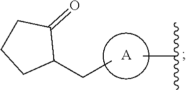
Z₁ and Z₂ are each independently hydrogen or straight or branched C1-C5 alkyl; and
A is a C3-C7 aryl group or a C3-C7 heteroaryl group.

13. A feed composition for preventing or ameliorating sarcopenia comprising a sesquiterpene derivative represented by Chemical Formula 1 below or a feed acceptable salt thereof as an active ingredient: 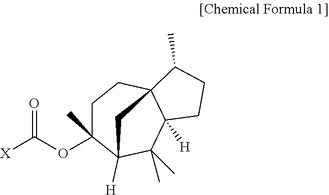
wherein,
X is 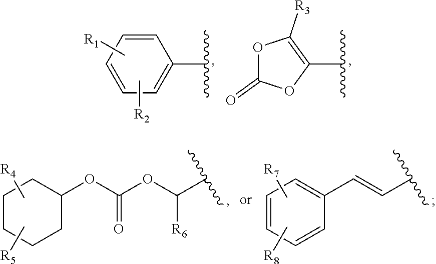
R₁ to R₈ are each independently hydrogen, halogen, straight or branched C1-C5 alkyl, straight or branched C2-C5 alkenyl, C2-C5 alkynyl, C3-C7 cycloalkyl, straight or branched C1-C5 alkoxy, —C(═O)Y₁, —OC(═O)Y₂, cyano, or hydroxyl, wherein one or more hydrogens of the C1-C5 alkyl, C3-C7 cycloalkyl, or C1-C5 alkoxy are each independently unsubstituted or substituted with one or more substituents selected from the group consisting of halogen, hydroxy, cyano, nitro, NY₃Y₄, and 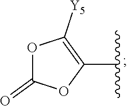
Y₁ to Y₅ are each independently hydrogen, straight or branched C1-C5 alkyl, or C3-C7 cycloalkyl, wherein one or more hydrogens of the C1-C5 alkyl or C3-C7 cycloalkyl are independently unsubstituted or substituted with one or more substituents selected from the group consisting of halogen, hydroxy, cyano, nitro, NZ₁Z₂, and 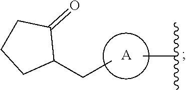
Z₁ and Z₂ are each independently hydrogen or straight or branched C1-C5 alkyl; and
A is a C3-C7 aryl group or a C3-C7 heteroaryl group.
